(19) United States
(12) Patent Application Publication
(10) Pub. No.: US 2020/0253926 A1
(43) Pub. Date: Aug. 13, 2020

(54) THERAPEUTIC COMBINATIONS

(71) Applicant: SUPERSALUS, INC., Carlsbad, CA (US)

(72) Inventors: Neil M. Cowen, Carlsbad, CA (US); Khaled Yamout, Carlsbad, CA (US)

(21) Appl. No.: 16/787,404

(22) Filed: Feb. 11, 2020

Related U.S. Application Data
(60) Provisional application No. 62/804,950, filed on Feb. 13, 2019.

(51) Int. Cl. A61K 31/401; A61K 31/4406; A61P 9/00

(52) U.S. Cl. CPC A61K 31/401; A61P 9/00; A61K 31/4406

(57) ABSTRACT

Methods of treatment include administering effective amounts of a nitrogen oxide (NO) donor (such as nicorandil) and a hydrogen sulfide (H₂S) releasing agent (such as zofenopril) to a subject in need thereof. In various embodiments, the H₂S releasing agent is administered in an amount that is effective to enhance the therapeutic efficacy of the NO donor. Coformulations of the H₂S releasing agent and the NO donor are provided that are suitable for treating a number of conditions. In various embodiments, treatments for conditions such as chronic kidney disease, Duchenne Muscular Dystrophy, Becker Muscular Dystrophy, familial dilated cardiomyopathy and/or idiopathic dilated cardiomyopathy are provided.

### INCORPORATION BY REFERENCE TO PRIORITY APPLICATION

This application claims the benefit of U.S. Provisional Application No. 62/804,950, filed on Feb. 13, 2019, which is hereby incorporated herein by reference in its entirety.

### BACKGROUND

### Field

This disclosure relates to coadministration of pharmaceutical actives which activate the ATP-sensitive potassium (K_{ATP}) channel, inhibit angiotensin converting enzyme, function as nitric oxide donors and function as hydrogen sulfide donors or releasing agents and coformulations of pharmaceutical actives which activate the K_{ATP} channel, inhibit angiotensin converting enzyme, function as nitric oxide donors and function as hydrogen sulfide donors or releasing agents. Also described are salts, cocrystals and other means of combining actives with these activities into a single pharmaceutical active ingredient. Also described are methods of using these co-administered pharmaceutical actives and these co-formulations to reduce cardiovascular disease and mortality risk in patients with end stage renal disease, in patients with dilated cardiomyopathy, and in patients who have experienced an acute myocardial infarction and the use of these co-formulations and co-administered products in the treatment of cardioskeletal myopathy. Finally, what is described is the further coadministration or coformulation of these actives with a beta-blocker to treat patients with congestive heart failure.

### Description of the Related Art

The following background is provided as an aid in understanding the invention and is not admitted to describe or constitute prior art to the invention.

The K_{ATP} channel is an octomeric ion channel consisting of 4 copies of the sulfonyl urea receptor (SURx) and 4 copies of an inwardly rectifying ion channel (Kir6.x). K_{ATP} channels are widely distributed and present in a number of tissues including, but not limited to cardiac, cardiovascular smooth muscle, skeletal muscle, adipocytes, pancreas, and various CNS tissues. Their activity is primarily regulated by adenine nucleotides, characteristically being activated by falling ATP and rising ADP levels. Thus, they link cellular metabolism with membrane excitability. The K_{ATP} channel exists in several different isotypes or subspecies defined by the specific version of SURx receptor and Kir6.x that comprise the channel. The channel that is typically present in cardiac tissue include the SUR2a receptor, cardiovascular smooth muscle includes the SUR2b receptor while SUR1 containing channels are found in the brain, pancreas, and adipocytes. In skeletal muscle, both SUR2b and SUR1 isotypes of the channel have been identified. In most excitatory cells the K_{ATP} channel is closed under normal physiological conditions and open when the tissue is metabolically compromised, as characterized by falling ATP levels. This promotes K+ efflux and cell hyperpolarization, thereby preventing voltage operated Ca2+ channels (VOCs) from opening. Activation of the K_{ATP} channel in cardiovascular smooth muscle has been shown to result in vasodilation. Activation of the channel has been shown to be protective of tissues under ischemia reperfusion stress. Maintenance of K_{ATP} channel potassium current can limit the extent of atrophy under various stress conditions. In addition to the isotypes of the channel that exist in the cell membrane, the K_{ATP} channel also is present in the mitochondrial inner membrane. Some the cardioprotective effects of agonizing the K_{ATP} channel are attributable to agonizing the mitochondrial isotype of the channel as are some of the benefits realized in animal models of Alzheimer's disease. Diazoxide, nicorandil, pinacidil and minoxidil are examples of K_{ATP} channel openers.

Nitric oxide donors may release NO by its spontaneous donation, by enzymatic oxidation or by chemical reaction. Nitric oxide donors, like nitroglycerin, have a principal pharmacological action of relaxation of vascular smooth muscle. Although venous effects predominate. NO donors produce, in a dose-related manner, dilation of both arterial and venous beds. This serves to regulate blood pressure, reduce stress on failing hearts and increase blood flow to the heart. NO has a variety of functions such as the release of prostanoids, inhibition of platelet aggregation, effect on angiogenesis, and production of oxygen free radicals. In addition to nitroglycerin, isosorbide mononitrate, nicorandil, glyceryl trinitrate and sodium nitroprusside are examples of NO donors.

Angiotensin converting enzyme inhibitors limit the conversion of angiotensin I to angiotensin II preventing the vasoconstricting effect of angiotensin II resulting in vasodilation and a reduction in blood pressure. ACE inhibitors are used in the management of hypertension, to manage progression of congestive heart failure and left ventricular dysfunction and to reduce cardiovascular death and morbidity in individuals who have suffered a myocardial infarction. Examples of ACE inhibitors include benzepril, captopril, enalopril, lisinopril, zofenopril and ramipril.

Hydrogen sulfide (H₂S) is a cytoprotective physiological signaling molecule that acts in concert with NO and carbon monoxide (CO) to maintain physiological homeostasis in both the heart and circulation. H₂S is produced in mammalian tissue by 3 tissue specific enzymes: cystathionine c-lyase (CSE), cystathionine b-synthase (CBS), and 3-mercaptopyruvate sulfur transferase (3-MST). Recent experimental evidence has shown that H₂S is a potent cardioprotective signaling molecule, and the administration of H₂S releasing agents or donors significantly attenuates the pathological consequences of myocardial ischemia/reperfusion injury (I/R) and heart failure. Examples of H₂S releasing agents include sulfide salts, Lawesson's reagent/analogs, 1,2-dithiole-3-thiones, and zofenopril.

Beta-blockers are competitive antagonists that block the receptor sites for endogenous catecholamines epinephrine and norepinephrine on beta-adrenergic receptors, and thereby reduce the effect of epinephrine on cardiovascular smooth muscle and cardiac tissue, lowering blood pressure and reducing heart rate, and the heart's demand for oxygen. There are 3 types of beta-adrenergic receptors, betas located mainly in the heart and kidneys, beta₂ located mainly in the lungs, gastrointestinal tract, liver, uterus, vascular smooth muscle, and skeletal muscle, and beta₃ located in fat cells.

Nicorandil is a nicotinamide nitrate used as an antianginal agent. It is unique among therapeutics used in the treatment of ischemia in that it has two modes of action. First, by agonizing K_{ATP} channels, nicorandil increases transmembrane potassium conductance and relaxes peripheral and coronary arterioles. Nicorandil is capable of agonizing the mitochondrial form of the K_{ATP} channel. In doing so, it exerts a tissue protective effect in the context of cellular stress from various causes. Second, with its nitrate moiety, functioning as an NO donor, nicorandil increases intracellular concentrations of cGMP, resulting in peripheral vein and coronary artery dilation. Thus, because of its ability to dilate arteries and veins, nicorandil maximizes coronary flow while concomitantly reducing myocardial work through reductions in afterload. For these reasons, nicorandil has been successful in managing angina pectoris. The K_{ATP} channel activation of nicorandil has broad ranging effects, some of which are attributable to its activation of the mitochondrial K_{ATP} channel. Oral formulations of nicorandil have been approved in various countries and regions to treat stable angina pectoris. Intravenous formulations have also been approved in some regions and used during percutaneous coronary intervention (PCI).

### SUMMARY

Various embodiments provide a pharmaceutical coformulation comprising a nitrogen oxide (NO) donor and a hydrogen sulfide (H₂S) releasing agent, wherein the H₂S releasing agent is present in the coformulation an amount that is effective to enhance the therapeutic efficacy of the NO donor.

Various embodiments provide a method of treating a subject, comprising identifying a subject having a chronic kidney disease (CKD); and administering therapeutically effective amounts of a NO donor and a H₂S releasing agent to the subject. In various embodiments, the CKD is stage 3 CKD, stage 4 CKD, or stage 5 CKD. In an embodiment, the stage 5 CKD is end stage renal disease (ESRD).

Various embodiments provide a method of treating a subject, comprising identifying a subject having Duchenne Muscular Dystrophy or Becker Muscular Dystrophy; and administering therapeutically effective amounts of a NO donor and a H₂S releasing agent to the subject.

Various embodiments provide a method of treating a subject, comprising identifying a subject having familial dilated cardiomyopathy or idiopathic dilated cardiomyopathy; and administering therapeutically effective amounts of a NO donor and a H₂S releasing agent to the subject.

These and other embodiments are described in greater detail below.

### BRIEF DESCRIPTION OF THE DRAWINGS

FIGS. 1a and 1b show an Indexed XRPD pattern and results for zofenopril calcium chloride.

FIG. 2 shows ¹H NMR results for zofenopril calcium chloride form A.

FIG. 3 shows DSC results for zofenopril calcium chloride form A.

FIG. 4 shows TGA results for zofenopril calcium chloride form A

FIGS. 5a and 5b show dynamic vapor sorption (DVS) isotherm results for zofenopril calcium chloride form A.

FIG. 6 shows an XRPD pattern following analysis of hygroscopicity of zofenopril calcium form A.

FIG. 7 shows an XRPD pattern from sample 3537-39-01 which is a mixture of zofenopril calcium form B and zofenopril calcium chloride form A.

FIG. 8 shows an XRPD pattern from sample 7615-27-04 which is a mixture of zofenopril calcium form B and zofenopril calcium chloride form A.

FIG. 9 shows an XRPD pattern for sample 7537-61-02.

FIG. 10 shows an XRPD pattern for zofenopril calcium chloride form A sample 7537-55-01.

FIG. 11 shows an XRPD pattern for zofenopril sodium material A (sample 7537-83-01).

FIGS. 12a and 12b show a DVS isotherm and results for zofenopril sodium material A (sample 7537-83-01).

FIG. 13 shows an XRPD pattern for zofenopril sodium material B (sample 7537-51-01).

FIG. 14 shows a comparison of XRPD profiles of zofenopril sodium material A and zofenopril hemisodium.

FIGS. 15a and 15b shows an indexed XRPD pattern and results for polymorph nicorandil material B.

FIG. 16 shows experimental and calculated XRPD results for nicorandil HCl form A.

FIGS. 17a and 17b show a DVS isotherm and results for nicorandil HCl form A.

FIG. 18 shows XRPD results of sample following DVS analysis of nicorandil HCl form A.

### DETAILED DESCRIPTION

Various embodiments provide pharmaceutical coformulations comprising a nitrogen oxide (NO) donor and a hydrogen sulfide (H₂S) releasing agent, and methods of using them to treat a variety of diseases and/or conditions as described elsewhere herein, including for example CKD, Duchenne Muscular Dystrophy, Becker Muscular Dystrophy, familial dilated cardiomyopathy and idiopathic dilated cardiomyopathy.

Surprisingly, pharmaceutical coformulations have now been developed in which the H₂S releasing agent is present in the coformulation an amount that is effective to enhance the therapeutic efficacy of the NO donor. In various embodiments the effects of coadministering the NO donor and H₂S releasing agent are synergistic. This invention is not limited by theory of operation and the mechanism by which the H₂S releasing agent enhances the therapeutic efficacy of the NO donor is not completely understood. However, a possible mechanism is described herein for the benefit of those skilled in the art. Pharmacologically, nicorandil acts as both a KATP channel opener and a nitrate or NO donor. It is capable of agonizing both the cell membrane and mitochondrial forms of the KATP channel.

Zofenopril is a sulfhydryl containing ACE inhibitor that is also a H₂S releasing agent. In studies in mice, zofenopril acts as a H₂S releasing agent directly elevating plasma and myocardial tissue H₂S levels compared to vehicle treated animals. The early H₂S increase following zofenopril administration appears to be due to direct release by zofenopril, which has also been shown to slowly release H₂S into solution in a cell-free assay. H₂S is generated in tissues through the action of either cystathionine β-synthase (CBS) or cystathionine γ-lyase (CSE) acting on L-cysteine as a substrate. Zofenopril treatment appears to increase CSE expression and thus upregulates endogenous H₂S generation as well. Ramipril, an ACE inhibitor lacking the sulfhydryl group did not elevate plasma or myocardial tissue H₂S levels in the same model.

Zofenopril may potentiate one or more activities of nicorandil. Zofenopril administration in mice significantly increased both plasma and myocardial tissue NO₂ levels compared with vehicle treated animals, and there was a trend towards increasing the activation of endothelial nitric oxide synthase (eNOS) as measured by increased phosphorylation of eNOS¹¹⁷⁷. ACE inhibition may directly increase NO₂ bioavailability by a bradykinin mediated pathway. In the same mouse study, ramipril had virtually no effect on plasma or myocardial tissue NO₂ concentrations. This indicates that the increased NO₂ bioavailability of zofenopril is associated with its H₂S releasing activity rather than ACE inhibition. Other H₂S releasing agents appear to increase eNOS expression or activity as well.

Thus, coadministration of zofenopril and nicorandil may increase NO₂ bioavailability beyond that achieved by nicorandil alone or by the coadministration of nicorandil with any other ACE inhibitor, improving any NO₂ mediated therapeutic response.

Several of the effects of H₂S appear to be mediated by KATP channel activation. KATP channel current can be measured directly as can the resulting cell membrane hyperpolarization. Exposure of rat aortic SMCs to H₂S results in a significantly increased amplitude of KATP channel current. This excitory effect of H₂S was fully manifested 3 minutes after application of H₂S and nullified immediately after washing H₂S from the bath solution. This suggests it is a direct effect of the application of H₂S. Similarly, the cell membrane was rapidly hyperpolarized after application of H₂S and was reversed 3-5 minutes after the application of glibenclamide, a KATP channel agonist.

The expression of the components of the KATP channel (SUR2b and Kir6.1) are downregulated in cardiovascular smooth muscle of spontaneously hypertensive rats. In this context, administration of H₂S increased vascular expression of SUR2b and Kir6.1. Thus, over longer-term administration, H₂S may increase KATP mediated responses by increased expression of the KATP channel subunits.

Intravenous injection of H₂S provokes a transient but significant decrease in mean arterial blood pressure. The H₂S-induced decrease in blood pressure was antagonized by glibenclamide a KATP channel antagonist.

High glucose has been shown to impair KATP channels in human vascular smooth muscle cells. High glucose exposure of H₉c2 cardiac cells results in cytotoxicity, apoptosis, oxidative stress and mitochondrial damage. These effects were ameliorated by treatment with a H₂S donor (NaHS), or either of two KATP channel agonists, diazoxide and pinacidil. Pretreatment of these cells with either a mitochondrial KATP channel antagonist (5-HD) or glibenclamide prior to NaHS treatment significantly diminished the cardioprotective effects, suggesting they are mediated by KATP channel activation.

In Langendorff-perfused rat hearts subjected to ischemia/reperfusion, the use of a H₂S releasing agent (4-carboxypheny isothiocyanate) significantly improved the post-ischemic recovery of myocardial functional parameters, limited tissue injury and reduced the post-ischemic release of norepinephrine and the incidence of ventricular arrhythmias. These effects were antagonized by 5-HD.

In perfused rat atria, NaHS, a H₂S donor, increased atrial natriuretic peptide (ANP) secretion and decreased atria pressure in a dose dependent manner, which was blocked by pretreatment with a KATP channel antagonist.

In isolated rat hearts, zofenopril had little effect on pre-ischemic coronary flow or on contractile function. Zofenopril significantly improved reperfusion function. This protective effect was complete reversed by both KATP channel antagonists, glyburide and 5-HD. Reperfusion end diastolic pressure (EDP) and lactate dehydrogenase (LDH) release were also significantly reduced and these protective effects were completely reversed by glyburide and 5-HD. Time to contracture (TTC) during global ischemia was used to evaluate the cardioprotective effective of chromakalim, and zofenopril. The effect of the drugs was tested individually and in combination in 3 ratios. The maximum increase in TTC was significantly higher in the hearts treated with the combination vs. those treated with either drug alone. Comparing the response for each of the 3 combinations relative to the theoretical line of additivity on an isobologram indicates that the two compounds interact to give a super-additive response. Chromakalim was able to induce a near maximal relaxation of aortic rings, while the maximal response to zofenopril was only 89%. Both glyburide and 5-HD shifted the relaxation response curve of zofenopril to the right suggesting that the relaxation was KATP channel mediated. Super-additivity was also observed for the combination of zofenopril and chromakalim on aortic ring relaxation. Competition studies with glyburide show that it is a competitive antagonist of chromakalim, but a non-competitive antagonist of zofenopril, consistent with chromakalim being a direct KATP channel agonist while the activity of zofenopril on the channel is mediated indirectly via H₂S.

Accordingly, it appears that an H₂S releasing agent may enhance the therapeutic efficacy of an NO donor. by potentiating both NO₂ mediated therapeutic responses to the NO donor and the KATP channel mediated responses to the NO donor, with the potential for synergistic super-additivity. For example, coadministration of nicorandil with zofenopril may improve therapeutic responses to nicorandil without increasing the frequency or severity of adverse events which are primarily associated with metabolites of nicorandil rather than its direct pharmacological action or off target effects and to provide effective ACE inhibition and the therapeutic benefits that it contributes in treated patients.

Provided herein are pharmaceutical coformulations of K_{ATP} channel activators with NO donors, ACE inhibitors and H₂S releasing agents and their use in the treatment of end stage renal disease patients treated with hemo- or peritoneal dialysis or following a kidney transplant. Such coformulations also have therapeutic value in the treatment of patients with Duchenne's, Beckers or other X-linked muscular dystrophies, Familial non-X-linked dilated cardiomyopathy, Barth syndrome, idiopathic dilated cardiomyopathy, and arrhythmogenic cardiomyopathy, and heart failure.

Also provided herein is coadministration of a K_{ATP} channel activator with a NO donor, an ACE inhibitor, and a H₂S releasing agent to a patient with end stage renal disease which is concomitantly treated with hemo- or peritoneal dialysis, or has received a kidney transplant.

Also provided herein is a crystalline solid which can be reproducibly made and can serve as an active pharmaceutical ingredient in a pharmaceutical formulation wherein the crystalline solid possesses K_{ATP} channel agonist activity, is a NO donor, is an ACE inhibitor and is a H₂S releasing agent.

Provided for is a pharmaceutical salt that combines the following in a single molecular entity: NO donor, KATP channel agonist, ACE inhibition and hydrogen sulfide releasing. The salt consists of nicorandil and zofenopril in a 1:1 stoichiometry.

Also provided are pharmaceutical formulations of the salt including extended release formulations. Also provided are coformulations of the salt with nicorandil. In one coformulation the ratio of nicorandil to the salt is about 1:10. In other coformulations the ratio of nicorandil to the salt is in the range of about 1:9 to about 1:5. In other coformulations the ratio of nicorandil to the salt is in the range of about 1:5 to 1:2. In another coformulation the ratio of nicorandil to the salt is the range of about 1:2 to about 1:1. In other coformulations the ratio of nicorandil to the salt is about 1.1:1 to about 2.9:1. In another coformulation the ratio of nicorandil to the salt is 3:1 to about 5:1.

Provided are methods of improving the solubility of a salt or cocrystal of zofenopril prior to its coformulation with nicorandil by one of the following techniques: (a) particle size reduction involving comminution, spray drying, or other micronising techniques, (b) use of an ion exchange resin, (c) use of inclusion complexes, for example cyclodextrin, (d) compaction with a solubilizing agent including a low viscosity hypromellose, low viscosity methylcellulose or similarly functioning excipient or combinations thereof, (e) association with a salt prior to formulation, (f) use of a solid dispersion, (g) use of a self-emulsifying system, (h) addition of one or more surfactants to the formulation, (i) use of nanoparticles, or (j) combinations of these approaches.

Further provided herein are controlled release pharmaceutical coformulations containing a NO donor, a K_{ATP} channel agonist, an ACE inhibitor and a hydrogen sulfide releasing pharmaceutical active ingredient, which include at least one component that substantially inhibits release of the pharmaceutical active ingredients from the formulation until after gastric transit is complete. As used herein, “substantially inhibits” means less than 20% release, more preferably at least less than 10% release, or even more preferably at least less than 5% release of the drug from the formulation during gastric transport. Release can be measured in a standard USP based in-vitro gastric dissolution assay in a calibrated dissolution apparatus. See e.g., U.S. Pharmacopeia, Chapter 711 (2016). Substantial inhibition of drug release during gastric transit is achieved by inclusion of a component in the formulation selected from the group consisting of: (a) a pH sensitive polymer or co-polymer applied as a compression coating on a tablet, (b) a pH sensitive polymer or co-polymer applied as a thin film on a tablet, (c) a pH sensitive polymer or co-polymer applied as a thin film to an encapsulation system, (d) a pH sensitive polymer or co-polymer applied to encapsulated microparticles, (e) a non-aqueous-soluble polymer or copolymer applied as a compression coating on a tablet, (f) a non-aqueous-soluble polymer or co-polymer applied as a thin film on a tablet, (g) a non-aqueous soluble polymer applied as a thin film to an encapsulation system, and (h) a non-aqueous soluble polymer applied to microparticles, wherein the pH sensitive polymer or co-polymer is resistant to degradation under acid conditions. Alternatively, substantial inhibition of drug release during gastric transport can also be achieved by incorporation of the formulation in an osmotic pump system, by use of systems controlled by ion exchange resins, or by combinations of any of the above approaches.

Also provided are salts of nicorandil which have utility as pharmaceutical active ingredients, including but not limited to HCl.

Also provided are active pharmaceutical ingredients involving zofenopril that are salts or simultaneously salts and co-crystals, including but limited to calcium chloride, sodium, acetic acid, citric acid, creatine, gentisic acid, L-glutamic acid, L-lactic acid, oxalic acid, succinic acid, tartaric acid or vanillin.

Also provided are extended release coformulations of salts of nicorandil and a form of zofenopril that is simultaneously a salt and a cocrystal.

Also provided herein are extended release pharmaceutical coformulations containing a NO donor, a K_{ATP} channel agonist, an ACE inhibitor and a hydrogen sulfide releasing pharmaceutical active ingredient, wherein the formulation includes at least one component that contributes to extended release of the pharmaceutical active ingredients over an prolonged period, e.g., over a period of 2-4 hours following administration, over a period of 4-8 hours following administration, or over a period of 8 to more than 24 hours following administration. These formulations are characterized in having one of the following components: (a) a pH sensitive polymeric coating, (b) a hydrogel coating, (c) a film coating that controls the rate of diffusion of the drug from a coated matrix, (d) an erodible matrix that controls rate of drug release, (e) polymer coated pellets, granules or microparticles of drug which can be further encapsulated or compressed into a tablet, (f) an osmotic pump system containing the drug, (g) a compression coated tablet form of the drug, (h) a hydrogel incorporated into the matrix or (i) combinations of any of the approaches of (a)-(h) above.

As used herein, an erodable matrix is the core of a tablet formulation that, upon exposure to a suitable aqueous environment, begins a process of disintegration which facilitates the release of drug from the matrix. The rate of release of drug from the tablet is controlled both by the solubility of the drug and the rate of disintegration of the matrix.

Also provided herein are extended release pharmaceutical coformulations containing a NO donor, a K_{ATP} channel agonist, an ACE inhibitor and a hydrogen sulfide releasing pharmaceutical active ingredient, wherein the formulation includes a hydrogel which provides stability for the tablet matrix over time, controls the rate of release from the hydrated tablet matrix, are bioadhesive and gastro-retentive. Such hydrogels include polyethylene oxide of various molecular weights from about 700,000 to about 7,000,000. These hydrogels also include alginate, carrageenan, chitosan, dextran, agarose, cellulose and derivatives including methyl cellulose (MC), hydroxypropyl cellulose (HPC), hydroxypropylmethyl cellulose (HPMC), and carboxymethyl cellulose (CMC); guar gum, pectin, PEG, PLA, acrylates, block copolymers, and PVA, and combinations of natural and synthetic polymers.

Also provided herein are extended release pharmaceutical coformulations containing a NO donor, a K_{ATP} channel agonist, an ACE inhibitor and a H₂S releasing pharmaceutical active ingredient, which include oral dosage forms comprising tablets, multilayer tablets (coated or uncoated), multicoated tablets, capsules, hard or soft gelatin capsules, pellets, powders, granules, colloidal dispersions, dispersions, solutions or suspensions and emulsions and the like.

In another embodiment, the extended release pharmaceutical formulation further comprises at least one pharmaceutically acceptable excipient selected from a group comprising lubricants, binding agents, fillers, preservatives, disintegrants, plasticizers, aromatic substances, or a mixture thereof.

Suitable fillers are selected from the group comprising starch, lactose, microcrystalline cellulose, carboxy cellulose sodium, sucrose. Suitable binding agents are selected from the group comprising povidone, hydroxypropylcellulose, hydroxypropylmethylcellulose, ethylcellulose, starch, gelatin, disaccharides, xylitol, sorbitol, mannitol, polyvinylpyrrolidone, and PEG. Suitable lubricants are selected from the group comprising collodial anhydrous silica, magnesium stearate, talc, sodium stearyl fumarate, PEG, glyceryl monostearate, glyceryl tribehenate, glyceryl dibehenate, sorbitan monostearate, sucrose monopalmitate, calcium stearate or zinc stearate. Suitable disintegrants are selected from the group comprising starch, pregelatinized starch, microcrystalline cellulose, sodium bicarbonate in combination with citric or tartaric acid, alginic acid, ion exchange resins, sodium carboxymethyl starch, sodium starch glycollate, croscarmellose sodium, crospovidone, and their mixtures. Suitable plasticizer are selected from the group comprising glycerin, polyethylene glycols, polyethylene glycol monomethyl ether, propylene glycol, acetyl tributlyl citrate, acetyl triethyl citrate, castor oil, diacetylated monoglycerides, dibutyl sebacate, diethyl phthalate, triacetin, tributy citrate, diethyl phthalate (DEP), triethyl citrate. Suitable glidants are selected from the group comprising colloidal silicon dioxide, silicon dioxide, magnesium trisilicate, powdered cellulose, starch, talc, tribasic calcium phosphate, lactose, stearates, dibasic calcium phosphate, magnesium carbonate, magnesium oxide, calcium silicate, silicon dioxide aerogels and their mixtures.

The tablet matrix may be coated with a functional or non-functional coating selected from sucrose, shellac, zein, HPMC, PVAP, CAP, waxes, latexes, cellulosics, vinyls, glycols, acrylics, fats, silicon elastomers, polyhydric alcohols, acetate esters, phthalate esters, glycerides, oils, and may contain fillers such as calcium carbonate, talc, or titanium dioxide, colorants including dyes, aluminum lakes, iron oxides or titanium dioxide, antiadhesives, flavors and surfactants.

The coating process may include spray coating, compression coating, dip coating, electrostatic coating, film coating, which may also include subcoating, lamination, binder solutions followed by dusting powders, polishing, enteric films, or extended release films.

Embodiments of formulations as described herein are useful upon oral administration to control release of the active(s) from the formulation for about 1-2 hours, 2-4 hours, 4-8 hours, 8-12 hours, 12-18 hours or over about 24 hours. Given the half-life of each active in circulation and the duration of activity which extends beyond the time period over which there are measureable circulating concentrations of each active, sustaining release from the formulation for 4-8 hours may be optimal for twice daily dosing and for 12-18 hours may be optimal for once per day dosing.

Also provided are pharmaceutical cocrystals of zofenopril and nicorandil with 1:1, 1:2, 1:3 or 1:4 stoichiometry. Crystallization conditions for such cocrystals include, but are not limited to evaporation, slurry, cools, vapor diffusion, solvent/antisolvent precipitation, combinations of these techniques as well as liquid-assisted grinding or co-melting.

The cocrystal with 1:4 stoichiometry is optimal for use in the treatment of ESRD patients while the cocrystal with 1:2 stoichiometry is optimal for the treatment of dilated cardiomyopathy. The use of a cocrystal of zofenopril and nicorandil may improve the solubility of zofenopril at pH 3-8 without reducing the solubility of nicorandil over the same pH range.

Also provided are pharmaceutical cocrystals of zofenopril and other cocrystal formers. Crystallization conditions for such cocrystals include, but are not limited to evaporation, slurry, cools, vapor diffusion, solvent/antisolvent precipitation, combinations of these techniques as well as liquid-assisted grinding or co-melting.

Cocrystal formers include, but are not limited to: salicylic acid, adipic acid, fumaric acid, succinic acid, acetic acid, maleic acid, 1-maleic acid, malonic acid, d/l-mandelic acid, nicotinamide, lactamide, benzamide, hippuric acid, benzoic acid, gentisic acid, 2,5-dihydrobenzoic acid, p-hydroxybenzoic acid, tartaric acid, oxalic acid, glutaric acid, citric acid, d/1-lactic acid, 1-lactic acid, sorbic acid, glycolic acid, stearic acid, 1,2-ethanedisulfonic acid naphthalenesulfonic acid, p-toluenesulfonic acid, s(+) camphor 10-sulfonic acid, glycine, 1-tryptophane, 1-leucine, 1-arginine, 1-lysine, 1-aspartic acid, 1-glutamic acid, glycolamide, tromethamine, n-methyl-D-glucamine, D-mannitol, lactose, sucrose, maltose, saccharine, creatine, ethylmaltol, 1-ascorbic acid, urea, isonicotinamide, homosynthon, ethylmalonic acid, 3,5-dinitrobenzoic acid, gallic acid, trigluoroacetic acid, pyrogallol, phloroglucinol, vanillin, vanillic acid, ferulic acid, caffeic acid, 4-amino salicylic acid, δ-valerolactam, 4,4′-bipyridine-N,N′-dioxide, 4-hydrorxybenzoic acid, hydrocinnamic acid, pimelic acid, acridine, and calcium chloride.

Nitrate donor drugs include, but are not limited to: nitroglycerin, isosorbide mononitrate, pentaerythrityl tetranitrate, sodium nitroprusside, nicorandil, S-nitroso-glutathione, S-nitroso-N-acetylpenicillamine, S-nitroso-N-valerylpenicillamine, or other S-nitrosothiol, aspirin, NCX4215, NCX4016, nipradilol, nitropravastatin, SNO-diclofenac, or other NO-NSAid, a diazeniumdiolate, SNO-captopril, SNO-t-PA or other S-nitroso hybrid molecules.

ACE inhibitors include, but are not limited to: captopril, enalapril, ramipril, quinapril, perindopril, lisinopril, benazepril, imidapril, zofenopril, trandolapril, cilazapril, and fosinopril.

K_{ATP} channel agonists include but are not limited to pinacidil, chromakalin, nicorandil and analogs thereof, a benzopyran, a cyanoguanidine, a thioformamide, a thiadiazine, a pyridyl nitrate, a cyclobutenedione, a dihydropyridine, a tertiary carbinol, a 3-trifluoromethyl-4-nitro-5-arylpyrazole, a thioamide, a dimethylchroman, a benzothiazole, a tetrahydrobenzothiazole, a benzenesulfonylurea, a benzenesulfonylthiourea, a benzenecarbonylurea, and a benzenecarbonylthiourea, or a pharmaceutically acceptable salt of any of the foregoing.

H₂S releasing agents include, but are not limited to a 1,2-dithiole-3-thione, a 1,2,4-thiadiazolidine-3,5-dione, 4-carboxyphenyl isothiocyanate, ACS-67, ACS-94, an acyl perthiol, AoAA, AP39, an arylthioamide, captopril, CaS, DAS, DADS, DATS, DATS-MSN, a dithioethione glycoconjugate, a dithioperoxyanhydride, DL-propargylglycine, GYY4137, IK-1001, an iminoester, an isothiscyanate glycoconjugate, Lawesson's reagent, a Lawesson's reagent analog, Na₂S, n-acetyl cysteine, NaSH, a N-benzoylthiobenzamide, an O-alkyl phosphorodithioate, an O-aryl phosphorodithioate, S-allylcysteine, a S-aroylthiooxine, SG-1002, S-propylargyl-cysteine, S-propylcysteine, YD0171, and zofenopril, or a pharmaceutically acceptable salt of any of the foregoing.

Provided herein are coformulations of pharmaceutical active which are NO donors, K_{ATP} channel agonists, H₂S generators, and ACE inhibitors which may be used to treat patients with ESRD. Use of these pharmaceutical formulations in these patients may reduce the likelihood of arrhythmias, reduce the rate of sudden cardiac death due to arrhythmia, reduce the likelihood of acute myocardial infarction, reduce the rate of death due to acute myocardial infarction, improve the likelihood of surviving an acute myocardial infarction, reduce the extent of myocardial damage as a result of acute myocardial infarction, reduce the extent of remodeling of the myocardium following acute myocardial infarction, reduce the progression to heart failure following acute myocardial infarction, slow or stop progression of heart failure, reduce death due to heart failure, reduce inflammation, reduce the level of reactive oxygen species, reduce the likelihood of thromboembolic events, reduce anemia, reduce death due to thromboembolic events, reduce cardiac microvascular dysfunction, stabilize atherosclerotic plaques, regress atherosclerotic plaque, reduce mitochondrial dysfunction, reduce cardiac myocyte apoptosis, reduce left ventricular hypertrophy, reduce left ventricular end systolic and diastolic volumes, improve left ventricular ejection fraction, reduce renal damage, improve renal function, and/or reduce overall mortality.

Also provided are pharmaceutical formulations of cocrystals of nicorandil and zofenopril or combinations of such cocrystals with nicorandil which may be used to treat patients with ESRD. Use of these pharmaceutical formulations in these patients may reduce the likelihood of arrhythmias, reduce the rate of sudden cardiac death due to arrhythmia, reduce the likelihood of acute myocardial infarction, reduce the rate of death due to acute myocardial infarction, improve the likelihood of surviving an acute myocardial infarction, reduce the extent of myocardial damage as a result of acute myocardial infarction, reduce the extent of remodeling of the myocardium following acute myocardial infarction, reduce the progression to heart failure following acute myocardial infarction, slow or stop progression of heart failure, reduce death due to heart failure, reduce inflammation, reduce the level of reactive oxygen species, reduce the likelihood of thromboembolic events, reduce anemia, reduce death due to thromboembolic events, reduce cardiac microvascular dysfunction, stabilize atherosclerotic plaques, regress atherosclerotic plaque, reduce mitochondrial dysfunction, reduce cardiac myocyte apoptosis, reduce left ventricular hypertrophy, reduce left ventricular end systolic and diastolic volumes, improve left ventricular ejection fraction, reduce renal damage, improve renal function, and/or reduce overall mortality.

Also provided are pharmaceutical coformulations of nicorandil or a pharmaceutically acceptable salt thereof and cocrystals of zofenopril and coformulations of nicorandil or a pharmaceutically acceptable salt thereof and a pharmaceutically acceptable salt of zofenopril which may be used to treat patients with ESRD. Use of these pharmaceutical formulations in these patients may reduce the likelihood of arrhythmias, reduce the rate of sudden cardiac death due to arrhythmia, reduce the likelihood of acute myocardial infarction, reduce the rate of death due to acute myocardial infarction, improve the likelihood of surviving an acute myocardial infarction, reduce the extent of myocardial damage as a result of acute myocardial infarction, reduce the extent of remodeling of the myocardium following acute myocardial infarction, reduce the progression to heart failure following acute myocardial infarction, slow or stop progression of heart failure, reduce death due to heart failure, reduce inflammation, reduce the level of reactive oxygen species, reduce the likelihood of thromboembolic events, reduce anemia, reduce death due to thromboembolic events, reduce cardiac microvascular dysfunction, stabilize atherosclerotic plaques, regress atherosclerotic plaque, reduce mitochondrial dysfunction, reduce cardiac myocyte apoptosis, reduce left ventricular hypertrophy, reduce left ventricular end systolic and diastolic volumes, improve left ventricular ejection fraction, reduce renal damage, improve renal function, and/or reduce overall mortality.

Also provided are coadministration of pharmaceutical formulations of cocrystals of nicorandil and zofenopril with a second pharmaceutical product to patients with ESRD. Such coadministered pharmaceutical products include, but are not limited to ion-removing agents, beta-blockers, antibiotics, analgesics and antipyretics, lipid lowering agents including, but not limited to statins, fenofibrate, omega-3 fatty acids, ezetimibe, and PCSK9 inhibitors, blood pressure lowering medications including, but not limited to ACE inhibitors, ARBs, diuretics, calcium channel blockers, renin-angiotensin-aldosterone system inhibitors, and combinations thereof, antiulcer agents and acid suppressants, antidepressants, antipsychotics, anxiolytics, sedatives and hypnotics, and other psychotherapeutic agents, anticonvulsants, cinacalcet, antithrombotic agents, antidiabetic agents, adrenals, anti-inflammatories, erythropoiesis stimulating agents, and antivirals.

Also provided is coadministration of nicorandil or a pharmaceutically acceptable salt thereof with an orally administered pharmaceutical formulation which upon absorption of the active and first pass metabolism gives rise to zofenoprilat. Also provided is the use of circulating zofenoprilat to potentiate each of the modes of action of nicorandil.

Also provided are coformulations of nicorandil or a pharmaceutically acceptable salt thereof, zofenopril or a pharmaceutically acceptable cocrystal or salt thereof, and a statin, and the use of these coformulations in subjects with coronary artery disease, atherosclerosis or arteriosclerosis. Administration of these coformulations to these patients may stabilize atherosclerotic plaque, regress atherosclerotic plaques, regress coronary artery disease, stabilize arteriosclerotic plaque, regress arteriosclerotic plaque, reduce the likelihood of arrhythmias, reduce the rate of sudden cardiac death due to arrhythmia, reduce the likelihood of acute myocardial infarction, reduce the rate of death due to acute myocardial infarction, improve the likelihood of surviving an acute myocardial infarction, reduce the extent of myocardial damage as a result of acute myocardial infarction, reduce the extent of remodeling of the myocardium following acute myocardial infarction, reduce the progression to heart failure following acute myocardial infarction, slow or stop progression of heart failure, reduce death due to heart failure, reduce inflammation, reduce the level of reactive oxygen species, reduce the likelihood of thromboembolic events, reduce anemia, reduce death due to thromboembolic events, reduce cardiac microvascular dysfunction, reduce mitochondrial dysfunction, reduce cardiac myocyte apoptosis, reduce left ventricular hypertrophy, reduce left ventricular end systolic and diastolic volumes, improve left ventricular ejection fraction, reduce renal damage, improve renal function, and/or reduce overall mortality.

Use of embodiments of the formulations described herein in a patient with Duchenne muscular dystrophy may limit the progression of, improve or resolve dilated cardiomyopathy.

Use of embodiments of the formulations as described herein in a patient with Becker muscular dystrophy may limit the progression of, improve or resolve dilated cardiomyopathy.

Use of embodiments of the formulations as described herein in a patient with Barth syndrome may limit the progression of, improve or resolve dilated cardiomyopathy.

Use of embodiments of the formulations as described herein in a patient with dystrophin deficient dilated cardiomyopathy may limit the progression of, improve or resolve dilated cardiomyopathy.

Use of embodiments of the formulations as described herein in a patient with familial dilated cardiomyopathy may limit the progression of, improve or resolve dilated cardiomyopathy.

Use of embodiments of the formulations as described herein in a patient with idiopathic dilated cardiomyopathy may limit the progression of, improve or resolve dilated cardiomyopathy.

Use of embodiments of pharmaceutical formulation as described herein in a patient with Duchenne muscular dystrophy may: (1) limit inflammatory or immune responses; (2) reduce or eliminate mitochondrial dysfunction; (3) decrease oxidative stress; (4) decrease fibrosis; (5) decrease apoptosis of myocytes; (6) reduce microvascular dysfunction; (7) reduce microvascular induced ischemia; (8) reduce cardiac wall stress; (9) reduce interstitial collagen deposition; (10) reduce the activity or expression of matrix metalloproteases; (11) reduce left ventricular dilation; (12) reduce or regress left ventricular remodeling; (13) reduce dysfunctional viable myocardium; (14) reduce heart failure; and/or (15) reduce atrophy.

Use of embodiments of pharmaceutical formulations as described herein in a patient with Becker muscular dystrophy may: (1) limit inflammatory or immune responses; (2) reduce or eliminate mitochondrial dysfunction; (3) decrease oxidative stress; (4) decrease fibrosis; (5) decrease apoptosis of myocytes; (6) reduce microvascular dysfunction; (7) reduce microvascular induced ischemia; (8) reduce cardiac wall stress; (9) reduce interstitial collagen deposition; (10) reduce the activity or expression of matrix metalloproteases; (11) reduce left ventricular dilation; (12) reduce or regress left ventricular remodeling; (13) reduce dysfunctional viable myocardium; (14) reduce heart failure; and/or (15) reduce atrophy.

Use of embodiments of pharmaceutical formulations as described herein in a patient with Barth syndrome may: (1) limit inflammatory or immune responses; (2) reduce or eliminate mitochondrial dysfunction; (3) decrease oxidative stress; (4) decrease fibrosis; (5) decrease apoptosis of myocytes; (6) reduce microvascular dysfunction; (7) reduce microvascular induced ischemia; (8) reduce cardiac wall stress; (9) reduce interstitial collagen deposition; (10) reduce the activity or expression of matrix metalloproteases; (11) reduce left ventricular dilation; (12) reduce or regress left ventricular remodeling; (13) reduce dysfunctional viable myocardium; (14) reduce heart failure; and/or (15) reduce atrophy.

Use of embodiments of pharmaceutical formulations as described herein in a patient with dystrophin deficient dilated cardiomyopathy may: (1) limit inflammatory or immune responses; (2) reduce or eliminate mitochondrial dysfunction; (3) decrease oxidative stress; (4) decrease fibrosis; (5) decrease apoptosis of myocytes; (6) reduce microvascular dysfunction; (7) reduce microvascular induced ischemia; (8) reduce cardiac wall stress; (9) reduce interstitial collagen deposition; (10) reduce the activity or expression of matrix metalloproteases; (11) reduce left ventricular dilation; (12) reduce or regress left ventricular remodeling; (13) reduce dysfunctional viable myocardium; (14) reduce heart failure; and/or (15) reduce atrophy.

Use of embodiments of pharmaceutical formulations as described herein in a patient with Familial dilated cardiomyopathy may: (1) limit inflammatory or immune responses; (2) reduce or eliminate mitochondrial dysfunction; (3) decrease oxidative stress; (4) decrease fibrosis; (5) decrease apoptosis of myocytes; (6) reduce microvascular dysfunction; (7) reduce microvascular induced ischemia; (8) reduce cardiac wall stress; (9) reduce interstitial collagen deposition; (10) reduce the activity or expression of matrix metalloproteases; (11) reduce left ventricular dilation; (12) reduce or regress left ventricular remodeling; (13) reduce dysfunctional viable myocardium; (14) reduce heart failure; and/or (15) reduce atrophy.

Use of embodiments of pharmaceutical formulations as described herein in a patient with idiopathic dilated cardiomyopathy may: (1) limit inflammatory or immune responses; (2) reduce or eliminate mitochondrial dysfunction; (3) decrease oxidative stress; (4) decrease fibrosis; (5) decrease apoptosis of myocytes; (6) reduce microvascular dysfunction; (7) reduce microvascular induced ischemia; (8) reduce cardiac wall stress; (9) reduce interstitial collagen deposition; (10) reduce the activity or expression of matrix metalloproteases; (11) reduce left ventricular dilation; (12) reduce or regress left ventricular remodeling; (13) reduce dysfunctional viable myocardium; (14) reduce heart failure; and/or (15) reduce atrophy.

Use of embodiments of pharmaceutical formulations as described herein in a patient with arrhythmogenic cardiomyopathy may: (1) reduce sudden cardiac death due to arrhythmic; (2) reduce cardiac arrest; (3) reduce ventricular arrhythmias; (4) reduce right ventricular dysfunction; (5) reduce biventricular dysfunction; (6) reduce or prevent progressive myocardial dystrophy with fibro-fatty replacement in the ventricular walls; (7) reduce abnormal cell-cell adhesions; (8) reduce myocyte death; and/or (9) reduced gap-junction and ion channel remodeling.

In the present context, the term “therapeutically effective” or “effective amount” indicates that the materials or amount of material is effective to prevent, alleviate, or condition, and/or to prolong the survival of the subject being treated.

The term “pharmaceutically acceptable” indicates that the identified material does not have properties that would cause a reasonably prudent medical practitioner to avoid administration of the material to a patient, taking into consideration the disease or conditions to be treated and the respective route of administration.

As used herein, the term “composition” refers to a formulation suitable for administration to an intended animal subject for therapeutic purposes; exemplary formulations contain at least one pharmaceutically active compound and at least one pharmaceutically acceptable carrier or excipient.

Other terms as used herein are defined below.

About: is used herein to mean in quantitative terms plus or minus 10%.

ACE inhibitor: Angiotensin converting enzyme inhibitors (ACE inhibitors) are medications that slow (inhibit) the activity of the enzyme ACE, which decreases the production of angiotensin II.

Acute myocardial infarction: myocardial infarction occurs when myocardial ischemia, a diminished blood supply to the heart, exceeds a critical threshold and overwhelms myocardial cellular repair mechanisms designed to maintain normal operating function and homeostasis. Ischemia at this critical threshold level for an extended period results in irreversible myocardial cell damage or death.

Adolescent: A person between 10 and 18 years of age.

Agonist: A chemical compound that has affinity for and stimulates physiological activity at cell receptors normally stimulated by naturally occurring substances, triggering a biochemical response. An agonist of a receptor can also be considered an activator of the receptor.

Amelioration of the symptoms of a particular disorder by administration of a particular pharmaceutical composition: refers to any lessening, whether permanent or temporary, lasting or transient that can be attributed to or associated with administration of the composition.

Analog: a compound that resembles another in structure but differs by at least one atom.

Antagonist: A substance that tends to nullify the action of another, as a drug that binds to a cell receptor without eliciting a biological response.

Apoptosis: A form of cell death in which a programmed sequence of events leads to the elimination of cells without releasing harmful substances into the surrounding area.

Arrhythmia: refers to any change from the normal sequence of cardiac electrical impulses. The electrical impulses may happen too fast, too slowly, or erratically—causing the heart to beat too fast, too slowly, or erratically. Arrhythmias include: atrial fibrillation, bradycardia, conduction disorders, premature contractions, tachycardia, and ventricular fibrillation.

Arrhythmogenic cardiomyopathy: is a rare, genetically-determined disease of the heart muscle characterized by a progressive myocardial dystrophy with fibro-fatty replacement functional and structural alterations of the right ventricle (RV), depolarization and repolarization abnormalities, arrhythmias with the left bundle branch block (LBBB) morphology and risk of sudden death.

Atherosclerotic Plaque: A buildup of cholesterol and fatty material within a blood vessel due to the effects of atherosclerosis.

Barth syndrome: a rare condition characterized by dilated cardiomyopathy, skeletal myopathy, recurrent infections due to neutropenia, and short stature. Mutations in the TAZ gene cause Barth syndrome which encodes tafazzin. Tafazzin is an enzyme involved in the modification of cardiolipin, which is critical in maintaining mitochondrial shape, energy production, and protein transport within cells. Dysfunctional mitochondria likely lead to other signs and symptoms of Barth syndrome.

Becker muscular dystrophy: is an inherited condition that causes progressive weakness and wasting of the skeletal and cardiac muscles. It primarily affects males. The age of onset and rate of progression can vary. Muscle weakness usually becomes apparent between the ages of 5 and 15. In some cases, cardiomyopathy is the first sign. BMD is caused by a mutation in the DMD gene and is inherited in an X-linked recessive manner. BMD is very similar to Duchenne muscular dystrophy, except that in BMD, symptoms begin later and progress at a slower rate.

Bilaminate: A component of a pharmaceutical dosage form that consists of the lamination of two distinct materials.

Bioavailability: Refers to the amount or extent of therapeutically active substance that is released from the drug product and becomes available in the body at the intended site of drug action. The amount or extent of drug released can be established by the pharmacokinetic-parameters, such as the area under the blood or plasma drug concentration-time curve (AUC) and the peak blood or plasma concentration (Cmax) of the drug.

Cardiac arrest: cardiac arrest is the sudden, unexpected loss of heart function, breathing and consciousness and usually results from an arrhythmia that disrupts the heart's pumping action, stopping blood flow to the rest of the body.

Cardioskeletal myopathy: Abnormalities of muscle cell structure and metabolism that lead to various patterns of weakness and dysfunction. The pathology extends to involve cardiac muscle fibers, resulting in a hypertrophic or dilated cardiomyopathy.

Cocrystal: solids that are crystalline single phase materials composed of two or more different molecular and/or ionic compounds generally in a stoichiometric ratio which are neither solvates nor simple salts.

Cocrystal former: a compound, typically not a pharmaceutical active, possessing properties that allows it under controlled conditions to form a crystalline single phase material with a pharmaceutical active.

Combination: refers to any association between or among two or more items. The combination can be two or more separate items, such as two compositions or two collections. It can be a mixture thereof, such as a single mixture of the two or more items, or any variation thereof.

Composition: refers to any mixture. It can be a solution, a suspension, liquid, powder, a paste, aqueous, non-aqueous or any combination thereof.

Compression tablet: Tablet formed by the exertion of pressure to a volume of tablet matrix in a die.

Compression coated tablet: A tablet formed by the addition of a coating by compression to a compressed core containing the pharmaceutical active.

Daily dosage: the total amount of a drug taken in a 24 hour period whether taken as a single dose or taken in multiple doses.

Derivative: a chemical substance derived from another substance by modification or substitution.

Dilated cardiomyopathy: is a condition in which the heart's ability to pump blood is decreased because the heart's main pumping chamber, the left ventricle, is enlarged and weakened. In some cases, it prevents the heart from relaxing and filling with blood as it should.

Duchenne muscular dystrophy: is a severe type of muscular dystrophy. The symptom of muscle weakness usually begins around the age of four in boys and worsens quickly. Typically, muscle loss occurs first in the upper legs and pelvis followed by those of the upper arms. This can result in trouble standing up. Most are unable to walk by the age of 12. Affected muscles may look larger due to increased fat content. Scoliosis is also common. Some may have intellectual disability. Females with a single copy of the defective gene may show mild symptoms. The disorder is X-linked recessive. It is caused by a mutation in the DMD gene which encodes the dystrophin protein. DMD affects about one in 5,000 males at birth.

Dystrophin: is a large rod-shaped cytoplasmic protein including 3685 amino acid residues. In skeletal and cardiac muscle, dystrophin associates with various proteins to form the dystrophin-associated protein complex (DAPC). The DAPC is thought to play a structural role in linking the actin cytoskeleton to the extracellular matrix, stabilizing the sarcolemma during repeated cycles of contraction and relaxation, and transmitting force generated in the muscle sarcomeres to the extracellular matrix

Encapsulation system: a structural feature that contains drug within such as a pharmaceutical capsule. A gel into which drug is incorporated also is considered an encapsulation system.

End stage renal disease: Chronic irreversible renal failure in which there is a need for renal replacement therapy in the form of dialysis or kidney transplant. Also known as Chronic Kidney Disease Stage 5 or CKD 5.

Equivalent amount: an amount of a derivative of a drug that in assays or upon administration to a subject produces an equal effect to a defined amount of the non-derivatized drug.

Familial dilated cardiomyopathy: familial dilated cardiomyopathy (familial DCM) can be identified in 20 to 35 percent of patients diagnosed with idiopathic dilated cardiomyopathy by clinical screening of family members. Most familial DCM is transmitted in an autosomal dominant inheritance pattern, although all inheritance patterns have been identified (autosomal recessive, and mitochondrial). Familial DCM genetic studies have identified mutations in more than 30 genes.

Glidant: An inactive component of a pharmaceutical formulation that prevents caking of the matrix during processing steps.

H₂S releasing agent: non-endogenous chemical compounds that increase the generation or release of H₂S.

Heart failure: is a complex clinical syndrome that results from any structural or functional impairment of ventricular filling or ejection of blood.

Hemodialysis: a medical procedure to remove fluid and waste products from the blood and to correct electrolyte imbalances. This is accomplished using a machine and a dialyzer, also referred to as an “artificial kidney.” Hemodialysis is used to treat both acute kidney failure and end stage renal disease.

Idiopathic dilated cardiomyopathy: dilated cardiomyopathy (DCM) is characterized by dilatation and systolic dysfunction of one or both ventricles. DCM is classified as idiopathic (idiopathic dilated cardiomyopathy, or IDC) when all usual clinically detectable, except genetic, causes have been excluded.

Inflammation: is part of the complex biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair. The five classical signs of inflammation are heat, pain, redness, swelling, and loss of function.

Ingredient of a pharmaceutical composition: refers to one or more materials used in the manufacture of a pharmaceutical composition. Ingredient can refer to an active ingredient (an agent) or to other materials in the compositions. Ingredients can include water and other solvents, salts, buffers, surfactants, water, non-aqueous solvents, and flavorings.

Ischemia: is a restriction in blood supply to tissues, causing a shortage of oxygen that is needed for cellular metabolism.

Ischemic injury: injury to tissue that results from a low oxygen state usually due to obstruction of the arterial blood supply or inadequate blood flow leading to hypoxia in the tissue.

Kit: refers to a packaged combination. A packaged combination can optionally include a label or labels, instructions and/or reagents for use with the combination.

Kir: Pore forming subunit of the K_{ATP} channel. Also known as the inwardly rectifying subunit of the K_{ATP} channel. Typically existing as Kir6.x and infrequently as Kir2.x subspecies.

K_{ATP} channel: An ATP sensitive potassium ion channel across the cell membrane formed by the association of 4 copies of a sulfonylurea receptor and 4 copies of a pore forming subunit Kir.

Lubricant: An inactive component of a pharmaceutical formulation that provides for the flow of materials in various processing steps, particularly tableting.

Microparticle: A small particulate formed in the process of developing pharmaceutical formulations that may be coated prior to producing the final dosage from.

NO donor: is a molecular carrier of NO which can be effectively used in clinical application to increase NO availability.

Osmotically controlled release: A pharmaceutical dosage form in which the release of the active drug is principally achieved by the hydration of a swellable component of the formulation.

Peritoneal dialysis: a form of dialysis that uses a combination of the lining of the peritoneal membrane (abdominal cavity) and a solution. Peritoneal dialysis may need to be carried out 4-6 times per day.

Pharmaceutical composition: refers a composition that contains an agent and one or more other ingredients that is formulated for administration to a subject. An agent refers to an active ingredient of a pharmaceutical composition. Typically active ingredients are active for treatment of a disease or condition. The pharmaceutically active agent can be referred to as “a pharmaceutical active” or an “active pharmaceutical ingredient.”

Pharmaceutical effect: refers to an effect observed upon administration of an agent intended for treatment of a disease or disorder or for amelioration of the symptoms thereof.

Pharmacodynamic: An effect mediated by drug action.

Pharmacokinetic: Relating to the absorption, distribution, metabolism and elimination of the drug in the body.

Polymorph: A compound that shares the same chemistry but a different crystal structure.

Prodrug: refers to a compound which, when metabolized, yields the desired active compound. Typically, the prodrug is inactive, or less active than the active compound, but may provide advantageous handling, administration, or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound.

Prolonged Administration (prolonged basis): Administration of a pharmaceutically acceptable formulation of a drug for 7 or more days. Typically, prolonged administration is for at least two weeks, preferably at least one month, and even more preferably at least two months (i.e. at least 8 weeks).

Release formulation (extended), (or “extended release formulation”): A formulation of pharmaceutical product that, upon administration to animals, provides for release of the active pharmaceutical over an extended period of time than provided by formulations of the same pharmaceutical active that result in rapid uptake. Similar terms are sustained release, prolonged-release, and slow-release. In all cases, the preparation, by definition, has a reduced rate of release of active substance.

Release formulation (delayed), (or “delayed release formulation”): Delayed-release products are modified-release, but are not extended-release. They involve the release of discrete amount(s) of drug sometime after drug administration, e.g. enteric-coated products, and exhibit a lag time during which little or no absorption occurs.

Revascularization: is the restoration of perfusion to a body part or organ that has suffered ischemia. It is typically accomplished by surgical means. Vascular bypass and angioplasty, which may also involve stent implantation, are the two primary means of revascularization.

Salt: the neutral, basic or acid compound formed by the union of an acid or an acid radical and a base or basic radical.

Solid oral dosage form: pharmaceutical formulations designed for oral administration including capsules and tablets.

Subject: refers to animals, including mammals, such as human beings.

Sulfonylurea receptor: A component of the K_{ATP} channel responsible for interaction with sulfonylurea, other K_{ATP} channel antagonists, diazoxide and other K_{ATP} channel agonists.

Tablet: Pharmaceutical dosage form that is produced by forming a volume of a matrix containing pharmaceutical active and excipients into a size and shape suitable for oral administration.

Treatment: means any manner in which the symptoms of a condition, disorder or disease or other indication, are ameliorated or otherwise beneficially altered.

In one example, a coformulation of nicorandil or a pharmaceutically acceptable salt thereof and a salt with 1:1 stoichiometry of zofenopril and nicorandil is produced with the following composition: 18.85% w/w nicorandil, 18.65% w/w nicorandil:zofenopril salt, 30% polyethylene oxide (WSR-303), 30.4% microcrystalline cellulose and 1.2% fumed silica and 0.9% magnesium stearate. The materials are blended until uniform and then compressed into a tablet. The formulation can be compressed into tablets with a uncoated core tablet weight of either 32 mg which contains about 6 mg of nicorandil and about 6 mg of nicorandil:zofenopril salt, 64 mg which contains about 12 mg of nicorandil and about 12 mg of nicorandil:zofenopril salt, or 128 mg which contains about 24 mg of nicorandil and about 24 mg of nicorandil:zofenopril salt. These core tablets are then coated with a non-functional coating, the color of which depends on the tablet weight.

In a second example, a coformulation of nicorandil or a pharmaceutically acceptable sat thereof and a salt with 1:1 stoichiometry of zofenopril and nicorandil is produced with the following composition: 12.7% w/w nicorandil, 37.3% w/w nicorandil:zofenopril salt, 30% polyethylene oxide (WSR-303), 17.9% microcrystalline cellulose and 1.2% fumed silica and 0.9% magnesium stearate. The materials are blended until uniform and then compressed into a tablet. The formulation can be compressed into tablets with a uncoated core tablet weight of either 32 mg which contains about 4 mg of nicorandil and about 12 mg of nicorandil:zofenopril salt, 64 mg which contains about 8.1 mg of nicorandil and about 32.9 mg of the salt, or 128 mg which contains about 16.25 mg of nicorandil and about 47.75 mg of nicorandil:zofenopril salt. These core tablets are then coated with a non-functional coating, the color of which depends on the tablet weight.

In a third example a coformulation of nicorandil or a pharmaceutically acceptable salt thereof and a zofenopril salt or cocrystal is produced with the following composition: 25% w/w nicorandil, 12.5% of a salt or cocrystal of zofenopril, 30% polyethylene oxide (WSR-303), 30.4% microcrystalline cellulose and 1.2% fumed silica and 0.9% magnesium stearate. The materials are blended until uniform and then compressed into a tablet. The formulation can be compressed into tablets with a uncoated core tablet weight of either about 32 mg which contains 8 mg of nicorandil and 4 mg of a salt or cocrystal of zofenopril, 64 mg which contains 16 mg of nicorandil and 8 mg of a salt or cocrystal of zofenopril, or 128 mg which contains 32 mg of nicorandil and 16 mg of a salt or cocrystal of zofenopril. These core tablets are then coated with a non-functional coating, the color of which depends on the tablet weight.

In a fourth example a coformulation of nicorandil or a pharmaceutically acceptable salt thereof and a salt or cocrystal of zofenopril is produced with the following composition: 25% w/w nicorandil, 25% of a salt or cocrystal of zofenopril, 30% polyethylene oxide (WSR-303), 17.9% microcrystalline cellulose and 1.2% fumed silica and 0.9% magnesium stearate. The materials are blended until uniform and then compressed into a tablet. The formulation can be compressed into tablets with a uncoated core tablet weight of either about 32 mg which contains 8 mg of nicorandil and 8 mg of a salt or cocrystal of zofenopril, 64 mg which contains 16 mg of nicorandil and 16 mg of a salt or cocrystal of zofenopril, or 128 mg which contains 32 mg of nicorandil and 32 mg of a salt or cocrystal of zofenopril. These core tablets are then coated with a non-functional coating, the color of which depends on the tablet weight.

In a fifth example a formulation of a 1:2 stoichiometry cocrystal of zofenopril and nicorandil is produced with the following composition: 50% w/w of the cocrystal, 30% polyethylene oxide (WSR-303), 17.9% microcrystalline cellulose and 1.2% fumed silica and 0.9% magnesium stearate. The materials are blended until uniform and then compressed into a tablet. The formulation can be compressed into tablets with an uncoated core tablet weight of either about 32 mg which contains 16 mg of the cocrystal, 64 mg which contains 32 mg of the cocrystal, or 128 mg which contains 64 mg of the cocrystal. These core tablets are then coated with a non-functional coating, the color of which depends on the tablet weight.

In another example, a cocrystal of nicorandil and zofenopril is formed by dissolving zofenopril in methanol that has been heated to 50° C. to the limits of its solubility and then dissolving an 2× or 4× excess of nicorandil in the heated saturated solution. The mixture is stirred for a period of about 6 hours and then cooled to 0-10° C. to drive the formation of the cocrystal.

### EMBODIMENTS

In one embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has coronary artery disease.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has coronary artery disease.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has coronary artery disease.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has diabetes mellitus.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has diabetes mellitus.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has diabetes mellitus.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has experienced at least one acute myocardial infarction.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has experienced at least one acute myocardial infarction.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has experienced at least one acute myocardial infarction.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has congestive heart failure.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has congestive heart failure.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has congestive heart failure.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has anemia that is treated with an erythropoiesis stimulating agent.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has anemia that is treated with an erythropoiesis stimulating agent.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has anemia that is treated with an erythropoiesis stimulating agent.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has valvular heart disease.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has valvular heart disease.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has valvular heart disease.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has experienced at least one cerebrovascular accident or transient ischemic attack.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has experienced at least one cerebrovascular accident or transient ischemic attack.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has experienced at least one cerebrovascular accident or transient ischemic attack.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has peripheral artery disease.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has peripheral artery disease.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has peripheral artery disease.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has atrial fibrillation.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has atrial fibrillation.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has atrial fibrillation.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has experienced cardiac arrest.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has experienced cardiac arrest.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has experienced cardiac arrest.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has experienced ventricular arrhythmia.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has experienced ventricular arrhythmia.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has experienced ventricular arrhythmia.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has experienced at least one venous thromboembolism or pulmonary embolism.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has experienced at least one venous thromboembolism or pulmonary embolism.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has experienced at least one venous thromboembolism or pulmonary embolism.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has been treated with percutaneous coronary intervention.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who been treated with percutaneous coronary intervention.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has been treated with percutaneous coronary intervention.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has been treated with a coronary artery bypass graft.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has been treated with a coronary artery bypass graft.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has been treated with a coronary artery bypass graft.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has been implanted with a defibrillator.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has been implanted with a defibrillator.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has been implanted with a defibrillator.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has had a stent implanted.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has had a stent implanted.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has had a stent implanted.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who has left ventricular hypertrophy.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who has left ventricular hypertrophy.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who has left ventricular hypertrophy.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who is also treated with a beta-blocker.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who is also treated with a beta-blocker.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who is also treated with a beta-blocker.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who is also treated with a statin.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who is also treated with a statin.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who is also treated with a statin.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who is also treated with a P2Y₁₂ inhibitor.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who is also treated with a P2Y₁₂ inhibitor.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who is also treated with a P2Y₁₂ inhibitor.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who is also treated with warfarin.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who is also treated with warfarin.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who is also treated with warfarin.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who is also treated with a direct oral anticoagulant.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who is also treated with a direct oral anticoagulant.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who is also treated with a direct oral anticoagulant.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who is also treated with an ACE inhibitor.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who is also treated with an ACE inhibitor.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who is also treated with an ACE inhibitor.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with hemodialysis who is also treated with an ARB.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient treated with peritoneal dialysis who is also treated with an ARB.

In another embodiment a pharmaceutical coformulation containing a nitrate donor, a K_{ATP} channel agonist, an ACE inhibitor and a hydrogen sulfide releasing pharmaceutical active ingredient is administered to an end stage renal disease patient who has received a kidney transplant who is also treated with an ARB.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has coronary artery disease.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has coronary artery disease.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has coronary artery disease.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has diabetes mellitus.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has diabetes mellitus.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has diabetes mellitus.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has experienced at least one acute myocardial infarction.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has experienced at least one acute myocardial infarction.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has experienced at least one acute myocardial infarction.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has congestive heart failure.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has congestive heart failure.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has congestive heart failure.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has anemia that is treated with an erythropoiesis stimulating agent.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has anemia that is treated with an erythropoiesis stimulating agent.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has anemia that is treated with an erythropoiesis stimulating agent.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has valvular heart disease.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has valvular heart disease.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has valvular heart disease.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has experienced at least one cerebrovascular accident or transient ischemic attack.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has experienced at least one cerebrovascular accident or transient ischemic attack.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has experienced at least one cerebrovascular accident or transient ischemic attack.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has peripheral artery disease.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has peripheral artery disease.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has peripheral artery disease.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has atrial fibrillation.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has atrial fibrillation.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has atrial fibrillation.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has experienced cardiac arrest.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has experienced cardiac arrest.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has experienced cardiac arrest.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has experienced ventricular arrhythmia.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has experienced ventricular arrhythmia.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has experienced ventricular arrhythmia.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has experienced at least one venous thromboembolism or pulmonary embolism.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has experienced at least one venous thromboembolism or pulmonary embolism.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has experienced at least one venous thromboembolism or pulmonary embolism.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has been treated with percutaneous coronary intervention.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who been treated with percutaneous coronary intervention.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has been treated with percutaneous coronary intervention.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has been treated with a coronary artery bypass graft.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has been treated with a coronary artery bypass graft.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has been treated with a coronary artery bypass graft.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has been implanted with a defibrillator.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has been implanted with a defibrillator.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has been implanted with a defibrillator.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has had a stent implanted.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has had a stent implanted.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has had a stent implanted.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who has left ventricular hypertrophy.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who has left ventricular hypertrophy.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who has had a left ventricular hypertrophy.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who is also treated with a beta-blocker.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who is also treated with a beta-blocker.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who is also treated with a beta-blocker.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who is also treated with a statin.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who is also treated with a statin.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who is also treated with a statin.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who is also treated with a P2Y₁₂ inhibitor.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who is also treated with a P2Y₁₂ inhibitor.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who is also treated with a P2Y₁₂ inhibitor.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who is also treated with warfarin.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who is also treated with warfarin.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who is also treated with warfarin.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who is also treated with a direct oral anticoagulant.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who is also treated with a direct oral anticoagulant.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who is also treated with a direct oral anticoagulant.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who is also treated with an ACE inhibitor.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who is also treated with an ACE inhibitor.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who is also treated with an ACE inhibitor.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with hemodialysis who is also treated with an ARB.

In another embodiment a pharmaceutical coformulation of nicorandil or a pharmaceutically acceptable salt thereof and zofenopril or a pharmaceutically acceptable salt or cocrystal thereof is administered to an end stage renal disease patient treated with peritoneal dialysis who is also treated with an ARB.

In another embodiment a pharmaceutical coformulation of nicorandil or a pharmaceutically acceptable salt thereof and zofenopril or a pharmaceutically acceptable salt or cocrystal thereof is administered to an end stage renal disease patient who has received a kidney transplant who is also treated with an ARB.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has coronary artery disease.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has coronary artery disease.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has coronary artery disease.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has diabetes mellitus.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has diabetes mellitus.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has diabetes mellitus.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has experienced at least one acute myocardial infarction.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has experienced at least one acute myocardial infarction.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has experienced at least one acute myocardial infarction.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has congestive heart failure.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has congestive heart failure.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has congestive heart failure.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has anemia that is treated with an erythropoiesis stimulating agent.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has anemia that is treated with an erythropoiesis stimulating agent.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has anemia that is treated with an erythropoiesis stimulating agent.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has valvular heart disease.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has valvular heart disease.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has valvular heart disease.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has experienced at least one cerebrovascular accident or transient ischemic attack.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has experienced at least one cerebrovascular accident or transient ischemic attack.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has experienced at least one cerebrovascular accident or transient ischemic attack.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has peripheral artery disease.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has peripheral artery disease.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has peripheral artery disease.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has atrial fibrillation.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has atrial fibrillation.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has atrial fibrillation.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has experienced cardiac arrest.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has experienced cardiac arrest.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has experienced cardiac arrest.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has experienced ventricular arrhythmia.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has experienced ventricular arrhythmia.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has experienced ventricular arrhythmia.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has experienced at least one venous thromboembolism or pulmonary embolism.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has experienced at least one venous thromboembolism or pulmonary embolism.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has experienced at least one venous thromboembolism or pulmonary embolism.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has been treated with percutaneous coronary intervention.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who been treated with percutaneous coronary intervention.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has been treated with percutaneous coronary intervention.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has been treated with a coronary artery bypass graft.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has been treated with a coronary artery bypass graft.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has been treated with a coronary artery bypass graft.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has been implanted with a defibrillator.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has been implanted with a defibrillator.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has been implanted with a defibrillator.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has had a stent implanted.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has had a stent implanted.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has had a stent implanted.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who has left ventricular hypertrophy.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who has left ventricular hypertrophy.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who has left ventricular hypertrophy.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who is also treated with a beta-blocker.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who is also treated with a beta-blocker.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who is also treated with a beta-blocker.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who is also treated with a statin.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who is also treated with a statin.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who is also treated with a statin.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who is also treated with a P2Y₁₂ inhibitor.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who is also treated with a P2Y₁₂ inhibitor.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who is also treated with a P2Y₁₂ inhibitor.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who is also treated with warfarin.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who is also treated with warfarin.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who is also treated with warfarin.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who is also treated with a direct oral anticoagulant.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who is also treated with a direct oral anticoagulant.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who is also treated with a direct oral anticoagulant.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who is also treated with an ACE inhibitor.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who is also treated with an ACE inhibitor.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who is also treated with an ACE inhibitor.

In another embodiment a pharmaceutical formulation of a cocrystal of nicorandil and zofenopril or a coformulation of nicorandil and a cocrystal of nicorandil and zofenopril is administered to an end stage renal disease patient treated with hemodialysis who is also treated with an ARB.

In another embodiment a pharmaceutical formulation of a cocrystal of nicorandil and zofenopril or a coformulation of nicorandil and a cocrystal of nicorandil and zofenopril is administered to an end stage renal disease patient treated with peritoneal dialysis who is also treated with an ARB.

In another embodiment a pharmaceutical formulation of a cocrystal of nicorandil and zofenopril or a coformulation of nicorandil and a cocrystal of nicorandil and zofenopril is administered to an end stage renal disease patient who has received a kidney transplant who is also treated with an ARB.

Beta-blockers include, but are not limited to: acebutolol, atenolol, betaxolol, bisoprolol, bucindolol, carteolol, carvedilol, celiprolol, esmolol, labetalol, metoprolol, nadolol, nebivolol, oxprenolol, penbutolol, pindolol, propranolol, sotalol, and timolol.

Statins include, but are not limited to: atorvastatin, cerivastatin, fluvastatin, lovastatin, mevastatin, pitavastatin, pravastatin, rosuvastatin, and simvastatin.

P2Y₁₂ inhibitors include, but are not limited to: clopidogrel, prasugrel, ticagrelor, and cangrelor.

Oral anticoagulants include, but are not limited to: dabigatran, rivaroxaban, and apixaban.

ACE inhibitors include, but are not limited to: captopril, enalapril, ramipril, quinapril, perindopril, lisinopril, benazepril, imidapril, trandolapril, cilazapril, fosinopril and zofenopril.

Angiotensin II receptor blocking drugs (ARBs), include, but are not limited to: azilsartan, candesartan, eprosartan. Irbesartan, losartan, olmesartan, telmisartan, and valsartan.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who has previously received AAV microdystrophin gene therapy.

In a second embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is receiving AAV microdystrophin gene therapy.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is anticipated to receive AAV microdystrophin gene therapy.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who has previously received mini-dystrophin gene therapy.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is receiving mini-dystrophin gene therapy.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is anticipated to receive mini-dystrophin gene therapy.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who has previously received follistatin gene therapy.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is receiving follistatin gene therapy.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is anticipated to receive follistatin gene therapy.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who has previously received GALGT2 gene therapy.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is receiving GALGT2 gene therapy.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is anticipated to receive mGALGT2 gene therapy.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is co-administered a product that is used to repair ryanodine receptor-mediated intracellular calcium leak.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is co-administered a product that is used to repair ryanodine receptor-mediated intracellular calcium leak.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is co-administered a product that is used to modulate stretch-activated calcium channels.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is co-administered a product that is used to modulate stretch-activated calcium channels.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is co-administered a product that is used to upregulate utrophin.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is co-administered a product that is used to upregulate utrophin.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is co-administered a product that is used to inhibit myostatin.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is co-administered a product that is used to inhibit myostatin.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is co-administered a product that is used to selectively modulate the androgen receptor.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is co-administered a product that is used to selectively modulate the androgen receptor.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is co-administered co-enzyme Q10.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is co-administered co-enzyme Q10.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is co-administered a product that is used as a membrane sealant.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is co-administered a product that is used as a membrane sealant.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is co-administered a product that is used to inhibit NF-κB.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is co-administered a product that is used to inhibit NF-κB.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is co-administered a product that is used to inhibit connective tissue growth factor.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is co-administered a product that is used to inhibit connective tissue growth factor.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is co-administered a product that is a steroid.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is co-administered a product that is a steroid.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is co-administered a product that is used to inhibit histone deacetylase.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is co-administered a product that is used to inhibit histone deacetylase.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who has been treated with induced pluripotent stem cells.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who has been treated with induced pluripotent stem cells.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is anticipated to be treated with induced pluripotent stem cells.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is anticipated to be treated with induced pluripotent stem cells.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who has been treated with an exon skipping therapeutic.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who has been treated with an exon-skipping therapeutic.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is anticipated to be treated with an exon-skipping therapeutic.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is anticipated to be treated with an exon-skipping therapeutic.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is treated with the combination of a non-steroidal anti-inflammatory drug and isosorbide dinitrate.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is treated with the combination of a non-steroidal anti-inflammatory drug and isosorbide dinitrate.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is treated with rhLAM-111.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is treated with rhLAM-111.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who has received myoblast transplantation.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who has received myoblast transplantation.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is treated with idebenone.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is treated with idebenone.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is treated with tadalafil.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is treated with tadalafil.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is treated with tamoxifen.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is treated with tamoxifen.

In another embodiment, a pharmaceutical co-formulation of nicorandil or a pharmaceutically acceptable salt thereof and zofenopril or a pharmaceutically acceptable salt or cocrystal thereof is administered to a Duchenne Muscular Dystrophy patient who is treated with a drug that is an aldosterone inhibitor.

In another embodiment, a pharmaceutical co-formulation of nicorandil or a pharmaceutically acceptable salt thereof and zofenopril or a pharmaceutically acceptable salt or cocrystal thereof is administered to a Becker Muscular Dystrophy patient who is treated with a drug that is an aldosterone inhibitor.

In another embodiment a pharmaceutical co-formulation of a KATP channel opener, a NO donor, an ACE inhibitor and an H₂S releasing agent is administered to a patient with familial dilated cardiomyopathy.

In another embodiment a pharmaceutical co-formulation of a K_{ATP} channel opener, a NO donor, an ACE inhibitor and an H₂S releasing agent is administered to a patient with idiopathic dilated cardiomyopathy.

In another embodiment a pharmaceutical co-formulation of nicorandil or a pharmaceutically acceptable salt thereof and zofenopril or a pharmaceutically acceptable salt or cocrystal thereof is administered to a patient with familial dilated cardiomyopathy.

In another embodiment a pharmaceutical co-formulation of nicorandil or a pharmaceutically acceptable salt thereof and zofenopril or a pharmaceutically acceptable salt or cocrystal thereof is administered to a patient with idiopathic dilated cardiomyopathy.

In another embodiment a pharmaceutical co-formulation of a cocrystal of nicorandil and zofenopril or a co-formulation of nicorandil and a cocrystal of nicorandil and zofenopril is administered to a patient with familial dilated cardiomyopathy.

In another embodiment a pharmaceutical co-formulation of a cocrystal of nicorandil and zofenopril or a co-formulation of nicorandil and a cocrystal of nicorandil and zofenopril is administered to a patient with idiopathic dilated cardiomyopathy.

In another embodiment a pharmaceutical co-formulation of a K_{ATP} channel opener, a NO donor, an ACE inhibitor and an H₂S releasing agent is administered to a patient with familial dilated cardiomyopathy who is also treated with an ACE inhibitor.

In another embodiment a pharmaceutical co-formulation of a K_{ATP} channel opener, a NO donor, an ACE inhibitor and an H₂S releasing agent is administered to a patient with idiopathic dilated cardiomyopathy who is also treated with an ACE inhibitor.

In another embodiment a pharmaceutical co-formulation of nicorandil or a pharmaceutically acceptable salt thereof and zofenopril or a pharmaceutically acceptable salt or cocrystal thereof is administered to a patient with familial dilated cardiomyopathy who is also treated with an ACE inhibitor.

In another embodiment a pharmaceutical co-formulation of nicorandil or a pharmaceutically acceptable salt thereof and zofenopril or a pharmaceutically acceptable salt or cocrystal thereof is administered to a patient with idiopathic dilated cardiomyopathy who is also treated with an ACE inhibitor.

In another embodiment a pharmaceutical co-formulation of a cocrystal of nicorandil and zofenopril or a co-formulation of nicorandil and a cocrystal of nicorandil and zofenopril is administered to a patient with familial dilated cardiomyopathy who is also treated with an ACE inhibitor.

In another embodiment a pharmaceutical co-formulation of a cocrystal of nicorandil and zofenopril or a co-formulation of nicorandil and a cocrystal of nicorandil and zofenopril is administered to a patient with idiopathic dilated cardiomyopathy who is also treated with an ACE inhibitor.

In another embodiment a pharmaceutical co-formulation of a K_{ATP} channel opener, a NO donor, an ACE inhibitor and an H₂S releasing agent is administered to a patient with familial dilated cardiomyopathy who is also treated with a beta blocker.

In another embodiment a pharmaceutical co-formulation of a K_{ATP} channel opener, a NO donor, an ACE inhibitor and an H₂S releasing agent is administered to a patient with idiopathic dilated cardiomyopathy who is also treated with a beta blocker.

In another embodiment a pharmaceutical co-formulation of nicorandil or a pharmaceutically acceptable salt thereof and zofenopril or a pharmaceutically acceptable salt or cocrystal thereof is administered to a patient with familial dilated cardiomyopathy who is also treated with a beta blocker.

In another embodiment a pharmaceutical co-formulation of nicorandil or a pharmaceutically acceptable salt thereof and zofenopril or a pharmaceutically acceptable salt or cocrystal thereof is administered to a patient with idiopathic dilated cardiomyopathy who is also treated with a beta blocker.

In another embodiment a pharmaceutical co-formulation of a cocrystal of nicorandil and zofenopril or a co-formulation of nicorandil and a cocrystal of nicorandil and zofenopril is administered to a patient with familial dilated cardiomyopathy who is also treated with a beta blocker.

In another embodiment a pharmaceutical co-formulation of a cocrystal of nicorandil and zofenopril or a co-formulation of nicorandil and a cocrystal of nicorandil and zofenopril is administered to a patient with idiopathic dilated cardiomyopathy who is also treated with a beta blocker.

In another embodiment a pharmaceutical co-formulation of a K_{ATP} channel opener, a NO donor, an ACE inhibitor and an H₂S releasing agent is administered to a patient with familial dilated cardiomyopathy who is also treated with a diuretic.

In another embodiment a pharmaceutical co-formulation of a K_{ATP} channel opener, a NO donor, an ACE inhibitor and an H₂S releasing agent is administered to a patient with idiopathic dilated cardiomyopathy who is also treated with a diuretic.

In another embodiment a pharmaceutical co-formulation of nicorandil or a pharmaceutically acceptable salt thereof and zofenopril or a pharmaceutically acceptable salt or cocrystal thereof is administered to a patient with familial dilated cardiomyopathy who is also treated with a diuretic.

In another embodiment a pharmaceutical co-formulation of nicorandil or a pharmaceutically acceptable salt thereof and zofenopril or a pharmaceutically acceptable salt or cocrystal thereof is administered to a patient with idiopathic dilated cardiomyopathy who is also treated with a diuretic.

In another embodiment a pharmaceutical co-formulation of a cocrystal of nicorandil and zofenopril or a co-formulation of nicorandil and a cocrystal of nicorandil and zofenopril is administered to a patient with familial dilated cardiomyopathy who is also treated with a diuretic.

In another embodiment a pharmaceutical co-formulation of a cocrystal of nicorandil and zofenopril or a co-formulation of nicorandil and a cocrystal of nicorandil and zofenopril is administered to a patient with idiopathic dilated cardiomyopathy who is also treated with a diuretic.

In another embodiment a pharmaceutical co-formulation of a K_{ATP} channel opener, a NO donor, an ACE inhibitor and an H₂S releasing agent is administered to a patient with familial dilated cardiomyopathy who is also treated with digoxin.

In another embodiment a pharmaceutical co-formulation of a K_{ATP} channel opener, a NO donor, an ACE inhibitor and an H₂S releasing agent is administered to a patient with idiopathic dilated cardiomyopathy who is also treated with digoxin.

In another embodiment a pharmaceutical co-formulation of nicorandil or a pharmaceutically acceptable salt thereof and zofenopril or a pharmaceutically acceptable salt or cocrystal thereof is administered to a patient with familial dilated cardiomyopathy who is also treated with digoxin.

In another embodiment a pharmaceutical co-formulation of nicorandil or a pharmaceutically acceptable salt thereof and zofenopril or a pharmaceutically acceptable salt or cocrystal thereof is administered to a patient with idiopathic dilated cardiomyopathy who is also treated with digoxin.

In another embodiment a pharmaceutical co-formulation of a cocrystal of nicorandil and zofenopril or a co-formulation of nicorandil and a cocrystal of nicorandil and zofenopril is administered to a patient with familial dilated cardiomyopathy who is also treated with digoxin.

In another embodiment a pharmaceutical co-formulation of a cocrystal of nicorandil and zofenopril or a co-formulation of nicorandil and a cocrystal of nicorandil and zofenopril is administered to a patient with idiopathic dilated cardiomyopathy who is also treated with digoxin.

In another embodiment a pharmaceutical co-formulation of a K_{ATP} channel opener, a NO donor, an ACE inhibitor and an H₂S releasing agent is administered to a patient with familial dilated cardiomyopathy who is also treated with an anti-platelet agent.

In another embodiment a pharmaceutical co-formulation of a K_{ATP} channel opener, a NO donor, an ACE inhibitor and an H₂S releasing agent is administered to a patient with idiopathic dilated cardiomyopathy who is also treated with an anti-platelet agent.

In another embodiment a pharmaceutical co-formulation of nicorandil or a pharmaceutically acceptable salt thereof and zofenopril or a pharmaceutically acceptable salt or cocrystal thereof is administered to a patient with familial dilated cardiomyopathy who is also treated with an anti-platelet agent.

In another embodiment a pharmaceutical co-formulation of nicorandil or a pharmaceutically acceptable salt thereof and zofenopril or a pharmaceutically acceptable salt or cocrystal thereof is administered to a patient with idiopathic dilated cardiomyopathy who is also treated with an anti-platelet agent.

In another embodiment a pharmaceutical co-formulation of a cocrystal of nicorandil and zofenopril or a co-formulation of nicorandil and a cocrystal of nicorandil and zofenopril is administered to a patient with familial dilated cardiomyopathy who is also treated with an anti-platelet agent.

In another embodiment a pharmaceutical co-formulation of a cocrystal of nicorandil and zofenopril or a co-formulation of nicorandil and a cocrystal of nicorandil and zofenopril is administered to a patient with idiopathic dilated cardiomyopathy who is also treated with an anti-platelet agent.

In another embodiment a pharmaceutical co-formulation of a K_{ATP} channel opener, a NO donor, an ACE inhibitor and an H₂S releasing agent is administered to a patient with familial dilated cardiomyopathy who also utilizes a biventricular pacemaker.

In another embodiment a pharmaceutical co-formulation of a K_{ATP} channel opener, a NO donor, an ACE inhibitor and an H₂S releasing agent is administered to a patient with idiopathic dilated cardiomyopathy who also utilizes a biventricular pacemaker.

In another embodiment a pharmaceutical co-formulation of nicorandil or a pharmaceutically acceptable salt thereof and zofenopril or a pharmaceutically acceptable salt or cocrystal thereof is administered to a patient with familial dilated cardiomyopathy who also utilizes a biventricular pacemaker.

In another embodiment a pharmaceutical co-formulation of nicorandil or a pharmaceutically acceptable salt thereof and zofenopril or a pharmaceutically acceptable salt or cocrystal thereof is administered to a patient with idiopathic dilated cardiomyopathy who also utilizes a biventricular pacemaker.

In another embodiment a pharmaceutical co-formulation of a cocrystal of nicorandil and zofenopril or a co-formulation of nicorandil and a cocrystal of nicorandil and zofenopril is administered to a patient with familial dilated cardiomyopathy who also utilizes a biventricular pacemaker.

In another embodiment a pharmaceutical co-formulation of a cocrystal of nicorandil and zofenopril or a co-formulation of nicorandil and a cocrystal of nicorandil and zofenopril is administered to a patient with idiopathic dilated cardiomyopathy who also utilizes a biventricular pacemaker.

In another embodiment a pharmaceutical co-formulation of a K_{ATP} channel opener, a NO donor, an ACE inhibitor and an H₂S releasing agent is administered to a patient with familial dilated cardiomyopathy who also utilizes an implanted cardioverter defibrillator.

In another embodiment a pharmaceutical co-formulation of a K_{ATP} channel opener, a NO donor, an ACE inhibitor and an H₂S releasing agent is administered to a patient with idiopathic dilated cardiomyopathy who also utilizes an implanted cardioverter defibrillator.

In another embodiment a pharmaceutical co-formulation of nicorandil or a pharmaceutically acceptable salt thereof and zofenopril or a pharmaceutically acceptable salt or cocrystal thereof is administered to a patient with familial dilated cardiomyopathy who also utilizes an implanted cardioverter defibrillator.

In another embodiment a pharmaceutical co-formulation of nicorandil or a pharmaceutically acceptable salt thereof and zofenopril or a pharmaceutically acceptable salt or cocrystal thereof is administered to a patient with idiopathic dilated cardiomyopathy who also utilizes an implanted cardioverter defibrillator.

In another embodiment a pharmaceutical co-formulation of a cocrystal of nicorandil and zofenopril or a co-formulation of nicorandil and a cocrystal of nicorandil and zofenopril is administered to a patient with familial dilated cardiomyopathy who also utilizes an implanted cardioverter defibrillator.

In another embodiment a pharmaceutical co-formulation of a cocrystal of nicorandil and zofenopril or a co-formulation of nicorandil and a cocrystal of nicorandil and zofenopril is administered to a patient with idiopathic dilated cardiomyopathy who also utilizes an implanted cardioverter defibrillator.

In another embodiment a pharmaceutical co-formulation of a K_{ATP} channel opener, a NO donor, an ACE inhibitor and an H₂S releasing agent is administered to a patient with familial dilated cardiomyopathy who also utilizes a left ventricular assist device.

In another embodiment a pharmaceutical co-formulation of a K_{ATP} channel opener, a NO donor, an ACE inhibitor and an H₂S releasing agent is administered to a patient with idiopathic dilated cardiomyopathy who also utilizes a left ventricular assist device.

In another embodiment a pharmaceutical co-formulation of nicorandil or a pharmaceutically acceptable salt thereof and zofenopril or a pharmaceutically acceptable salt or cocrystal thereof is administered to a patient with familial dilated cardiomyopathy who also utilizes a left ventricular assist device.

In another embodiment a pharmaceutical co-formulation of nicorandil or a pharmaceutically acceptable salt thereof and zofenopril or a pharmaceutically acceptable salt or cocrystal thereof is administered to a patient with idiopathic dilated cardiomyopathy who also utilizes a left ventricular assist device.

In another embodiment a pharmaceutical co-formulation of a cocrystal of nicorandil and zofenopril or a co-formulation of nicorandil and a cocrystal of nicorandil and zofenopril is administered to a patient with familial dilated cardiomyopathy who also utilizes a left ventricular assist device.

In another embodiment a pharmaceutical co-formulation of a cocrystal of nicorandil and zofenopril or a co-formulation of nicorandil and a cocrystal of nicorandil and zofenopril is administered to a patient with idiopathic dilated cardiomyopathy who also utilizes a left ventricular assist device.

In another embodiment a pharmaceutical co-formulation of a K_{ATP} channel opener, a NO donor, an ACE inhibitor and an H₂S releasing agent is administered to a patient with familial dilated cardiomyopathy who is also treated with a modulator of cardiac myosin.

In another embodiment a pharmaceutical co-formulation of a K_{ATP} channel opener, a NO donor, an ACE inhibitor and an H₂S releasing agent is administered to a patient with idiopathic dilated cardiomyopathy who is also treated with a modulator of cardiac myosin.

In another embodiment a pharmaceutical co-formulation of nicorandil or a pharmaceutically acceptable salt thereof and zofenopril or a pharmaceutically acceptable salt or cocrystal thereof is administered to a patient with familial dilated cardiomyopathy who is also treated with a modulator of cardiac myosin.

In another embodiment a pharmaceutical co-formulation of nicorandil or a pharmaceutically acceptable salt thereof and zofenopril or a pharmaceutically acceptable salt or cocrystal thereof is administered to a patient with idiopathic dilated cardiomyopathy who is also treated with a modulator of cardiac myosin.

In another embodiment a pharmaceutical co-formulation of a cocrystal of nicorandil and zofenopril or a co-formulation of nicorandil and a cocrystal of nicorandil and zofenopril is administered to a patient with familial dilated cardiomyopathy who is also treated with a modulator of cardiac myosin.

In another embodiment a pharmaceutical co-formulation of a cocrystal of nicorandil and zofenopril or a co-formulation of nicorandil and a cocrystal of nicorandil and zofenopril is administered to a patient with idiopathic dilated cardiomyopathy who is also treated with a modulator of cardiac myosin.

In another embodiment, a pharmaceutical coformulation of a K_{ATP} channel opener, a NO donor, an ACE inhibitor, an H₂S releasing agent, and a beta-blocker is administered to a patient with congestive heart failure.

In another embodiment, a pharmaceutical co-formulation of nicorandil or a pharmaceutically acceptable salt thereof and zofenopril or a pharmaceutically acceptable salt or cocrystal thereof with a beta-blocker is administered to a patient with congestive heart failure.

In another embodiment a pharmaceutical co-formulation of a cocrystal of nicorandil and zofenopril combined with a beta blocker or a co-formulation of nicorandil and a cocrystal of nicorandil and zofenopril combined with a beta blocker is administered to a patient congestive heart failure.

### EXAMPLES

### Synthesis and Characterization of Zofenopril Calcium Chloride

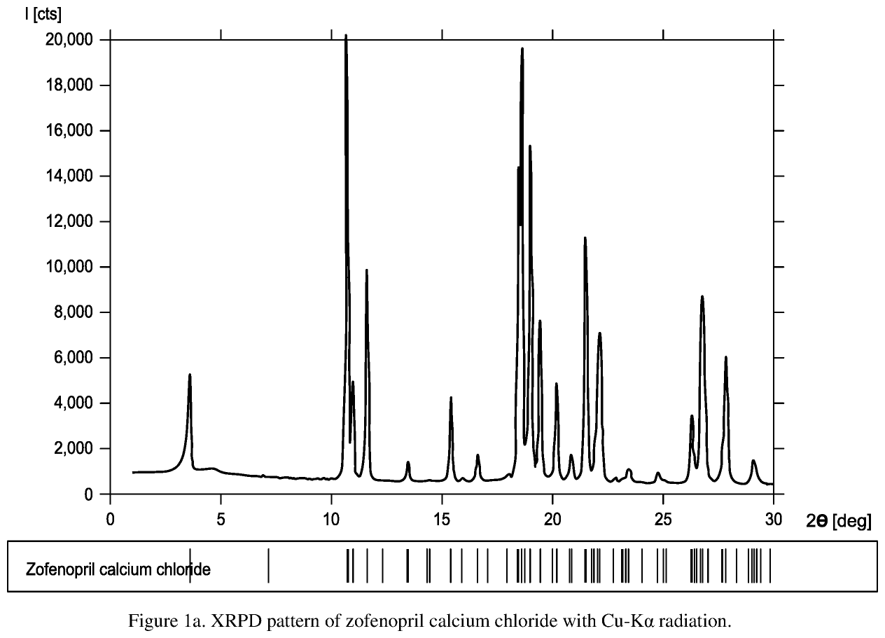

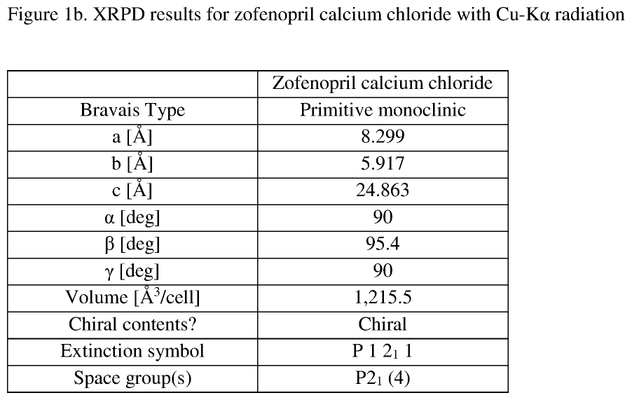

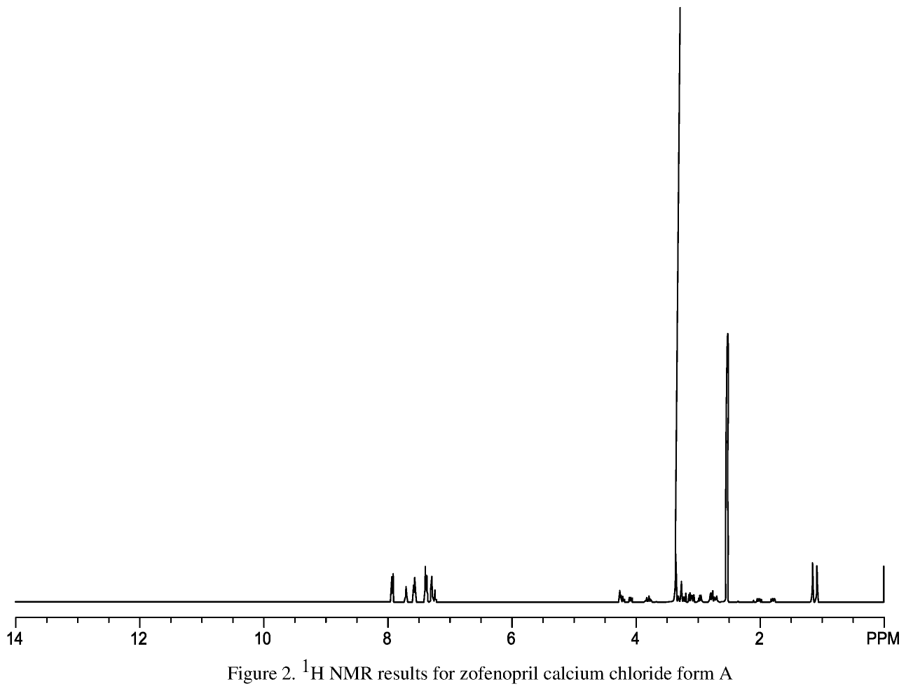

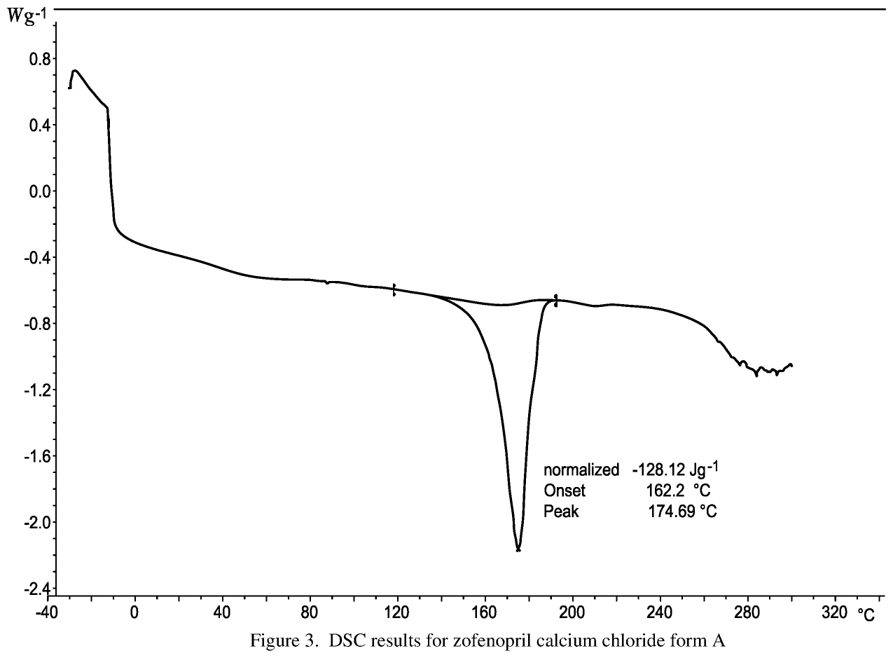

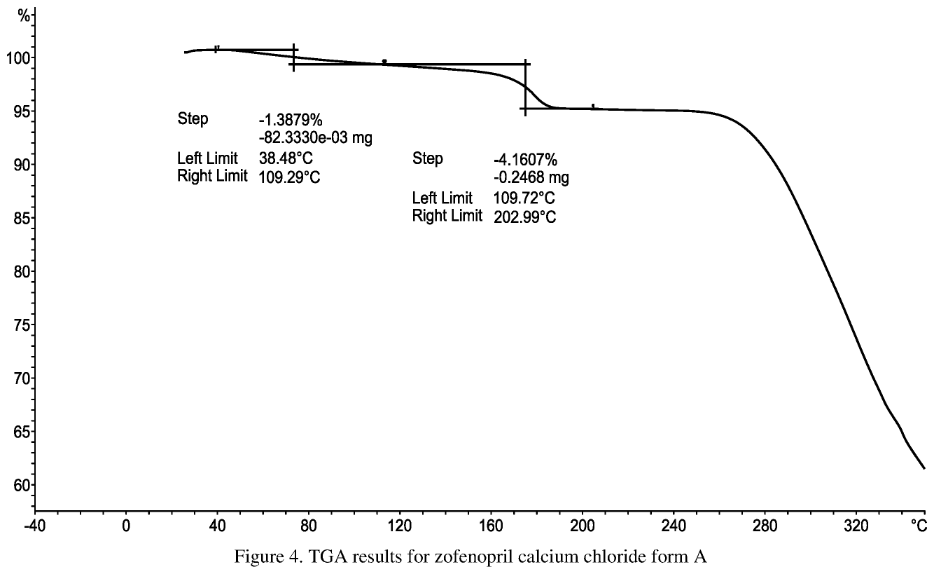

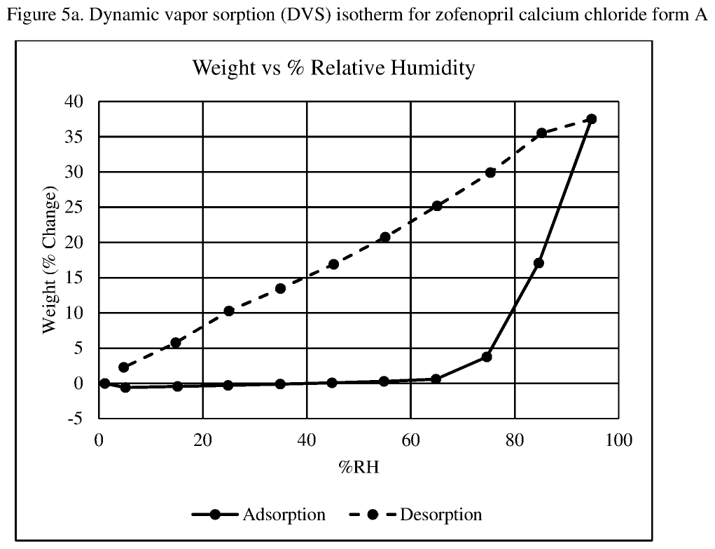

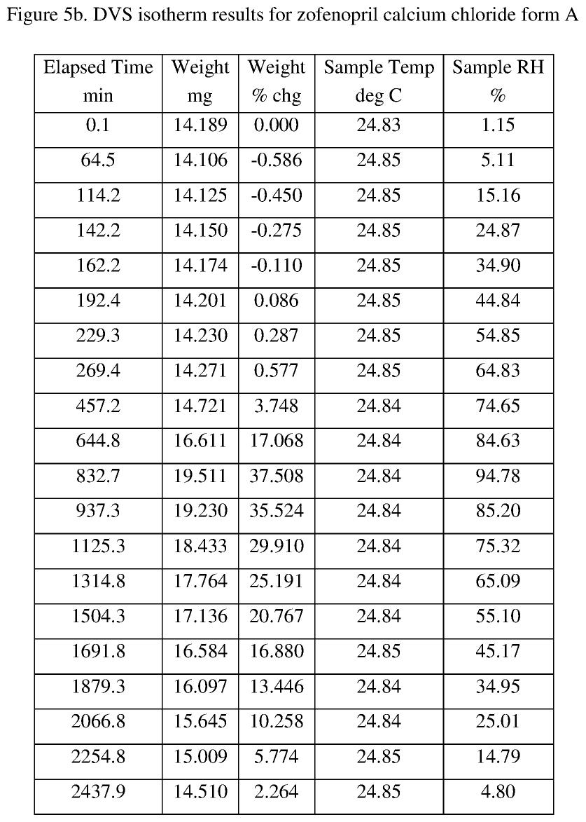

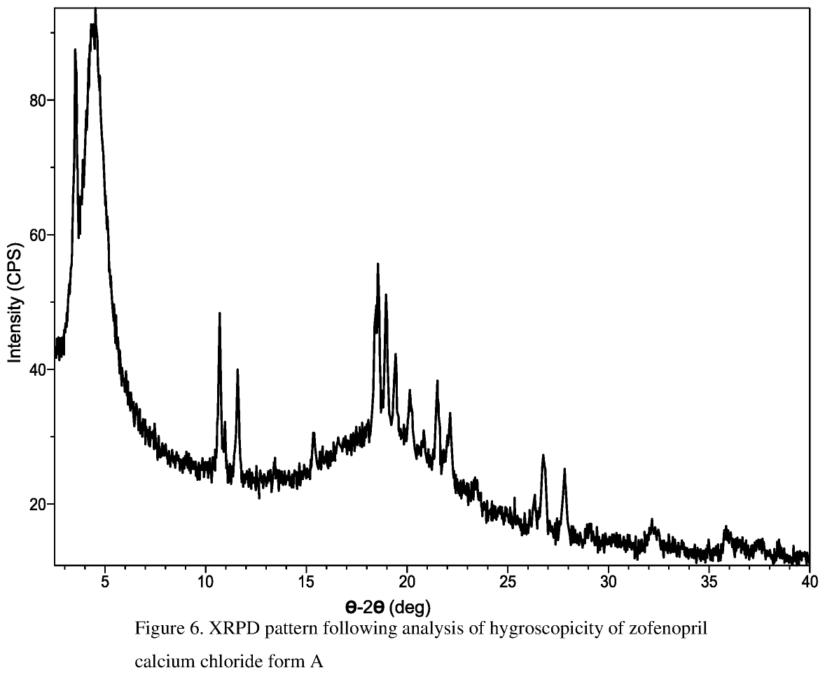

Zofenopril calcium was slurried in ACN. Two molar equivalents of HCl were added. The materials were allowed to slurry overnight. This resulted in a limited amount of white fine crystals which could be collected on 0.2 μm filter paper and dried under N₂. The resulting crystals were shown to be zofenopril calcium chloride form A and designated sample 7535-40-02. The indexed XRPD pattern for the material is shown in FIGS. 1a and 1b. ¹H NMR (DMSO-d₆) results were obtained for zofenopril calcium chloride form A sample 7537-40-02 and shown to be consistent with zofenopril with trace amounts of nicorandil and ACN (FIG. 2). DSC for zofenopril calcium chloride is shown as FIG. 3. On DSC this material has an onset at 162° C. This is in contrast to DSC results for zofenopril calcium which has an onset of 250.3° C. TGA results for zofenopril calcium chloride are provided in FIG. 4, there is 1.4% weight loss from 38 to 109° C. and 4.2% weight loss from 110 to 203° C. Based on IC, the calcium to zofenopril ratio is 0.99 to 1, and the chloride to zofenopril ratio is 0.89 to 1, which are consistent with 1:1:1 zofenopril calcium chloride. Hygroscopicity of zofenopril calcium chloride was evaluated by dynamic vapor sorption (DVS) and shown in FIGS. 5a and 5b. DVS analysis indicates that Zofenopril Calcium Chloride Form A exhibits limited hygroscopicity at or below 65% RH; however, the material may deliquesce if exposed to relative humidity conditions above 65% RH. On the sorption step up to 65% RH, the material gained 1.2% weight. Above this RH, the material gained an additional 36.9% weight. It should be noted that equilibration timed out at all steps above 65% RH, indicating that the material may pick up more moisture than what was measured if it was allowed a longer equilibration time. Most of the weight was lost on desorption with significant hysteresis. The material recovered after the DVS experiment was identified as Zofenopril Calcium Chloride Form A by XRPD (FIG. 6). Zofenopril calcium chloride form A exhibits limited aqueous solubility of approximately 0.1 mg/ml based on a gravimetric estimation.

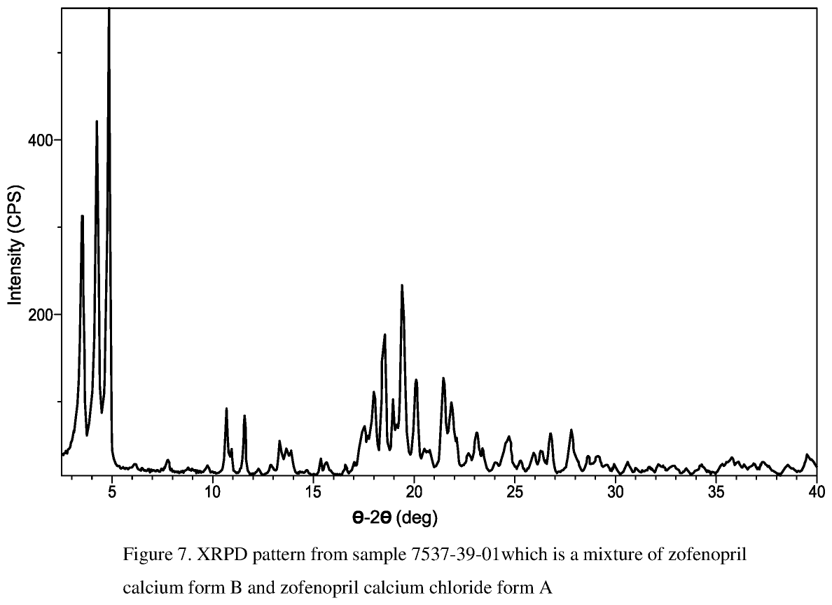

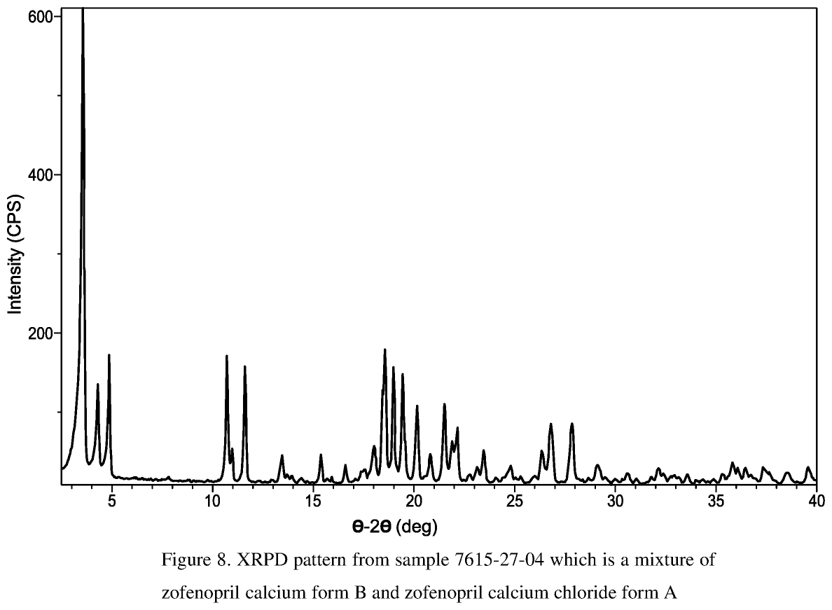

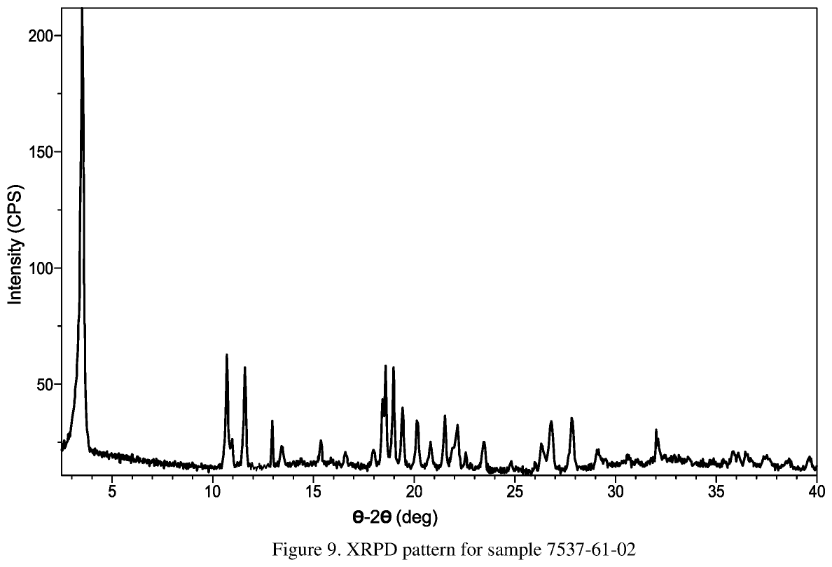

Zofenopril calcium chloride form A, typically in admixture with other materials, was also obtained from a series of reactions involving one molar equivalent of zofenopril calcium with one or two molar equivalents of nicorandil which were combined in ACN followed by the addition of one or two molar equivalents of HCl. The results were slurried at least 8 hours, but also for as long as 3 days. The results were filtered and dried under N₂. These reactions gave rise either to zofenopril calcium chloride form A or a combination of zofenopril calcium form A with zofenopril calcium form B. XRPD results for a reaction involving one molar equivalents of zofenopril calcium, nicorandil and HCl with the reaction slurried overnight before filtering giving rise to sample 7537-39-01 are shown in FIG. 7. XRPD results of a reaction involving one molar equivalent of zofenopril calcium, two molar equivalents of nicorandil and two molar equivalents of HCl and slurried for 3 days giving rise to sample 7615-27-04 are shown in FIG. 8. Zofenopril calcium chloride form A was also obtained as an admixture with CaCl₂*6(H₂O). This resulted from the reaction of zofenopril calcium in EtOAc with 2 molar equivalents of HCl. The reactants were slurried under ambient conditions for 3 days and then filtered and dried under N2 resulting in sample 7537-61-02. The XRPD results for this sample are shown in FIG. 9.

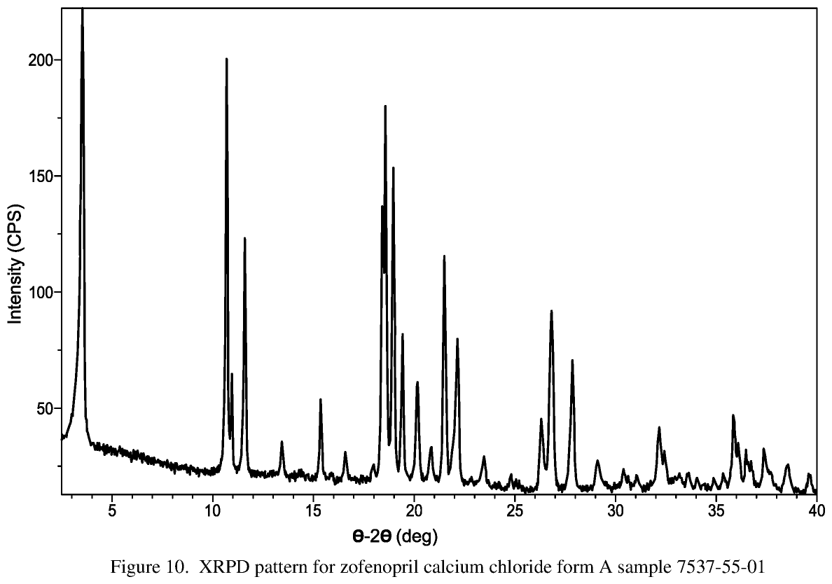

Similarly, zofenopril calcium form A was obtained from the reaction of zofenopril calcium, dissolved in ACN and reacted with 2 molar equivalents of HCl. The reactants were slurried overnight and then filtered and dried under N₂ yielding sample 7537-55-01. The XRPD results for this sample are provided as FIG. 10.

### Synthesis and Characterization of Zofenopril Sodium

Zofenopril calcium was slurried in water. One molar equivalent of H₂SO₄ was added stepwise at which point the material clumped and then broke as a flocculent. The resulting material was extracted with DCM. The resulting materials formed an oil which was washed with water. The resulting oil was warmed to 40° C. and dried over MgSO₄. The oil miscibilized upon warming. The resulting dried oil was placed under vacuum to remove any further solvent.

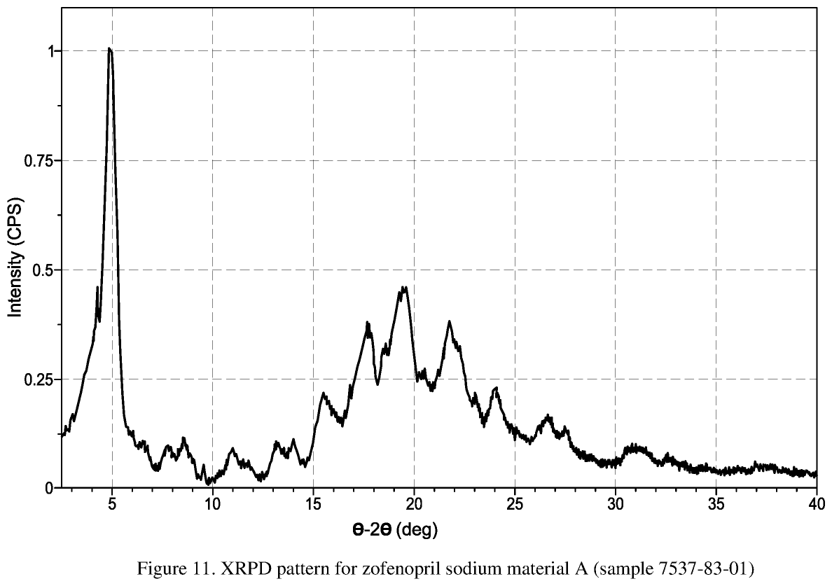

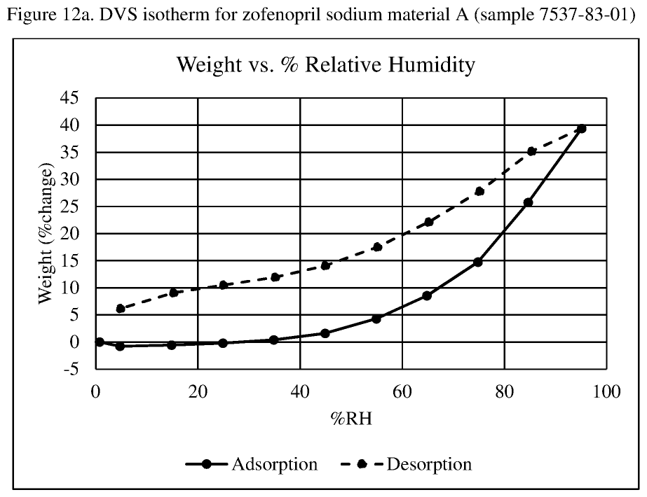

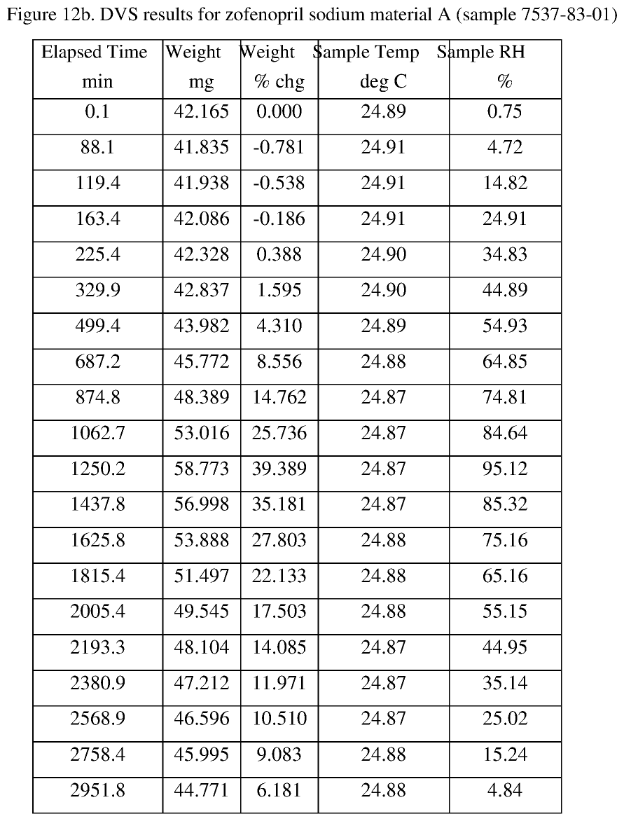

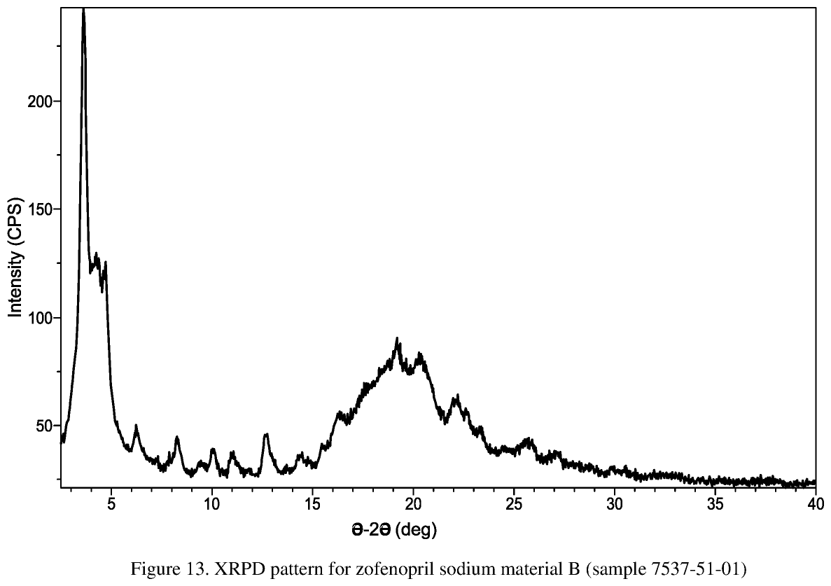

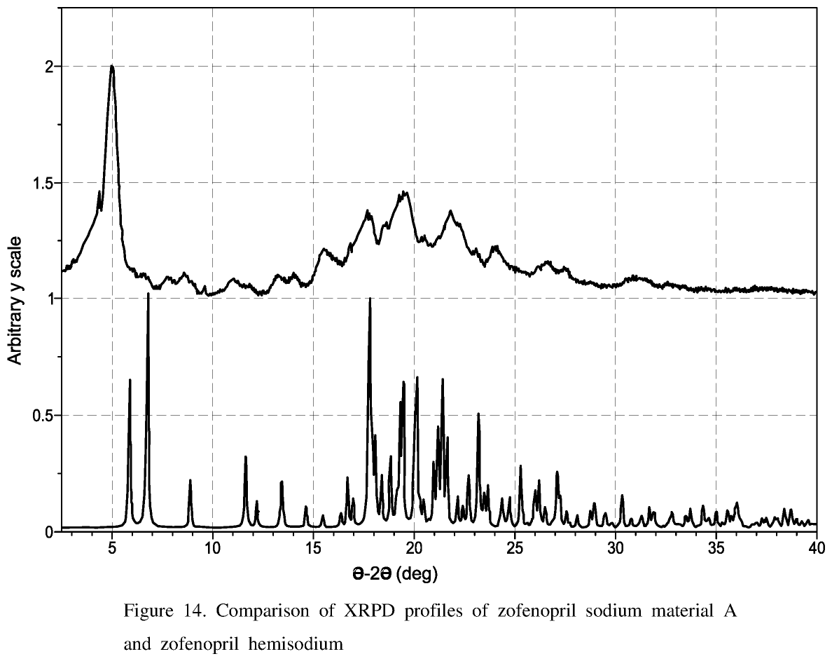

This oil, which is zofenopril free acid, was dissolved in ACN. NaOH in water was added to the dissolved zofenopril free acid solution. This resulted in the formation of a gel and a few white clumps. Additional water was added to the mixture at which point the gel broke and dissolved and a precipitate dropped out of solution. Additional ACN was added to this material and the resulting material was filtered and then rinsed with additional ACN resulting in a wet paste which was dried under N₂ yielding a free flowing powder designated zofenopril sodium material A (sample 7537-83-01). XRPD results for zofenopril sodium material A are provided in FIG. 11. The solubility of zofenopril sodium material A was tested and shown to be >48 mg/ml. The pH of the solution at the end of the solubility was shown to be 10. The hygroscopicity of zofenopril sodium material A was tested using DVS. The results of that test are provided in FIGS. 12a and 12b. The sample recovered following the DVS evaluation of zofenopril sodium material A was tested and shown to have a distinct XRPD profile to that of zofenopril sodium material A and was designed zofenopril sodium material B (FIG. 13 sample 7578-51-01). The XRPD profiles of zofenopril sodium material A and material B are distinct from that of zofenopril hemisodium (FIG. 14).

### Synthesis and Characterization of Nicorandil Material B

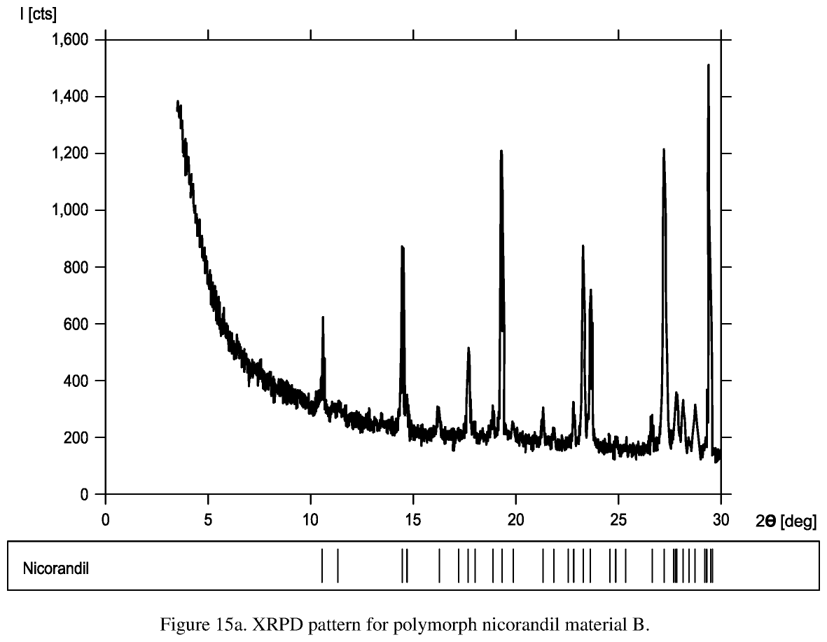

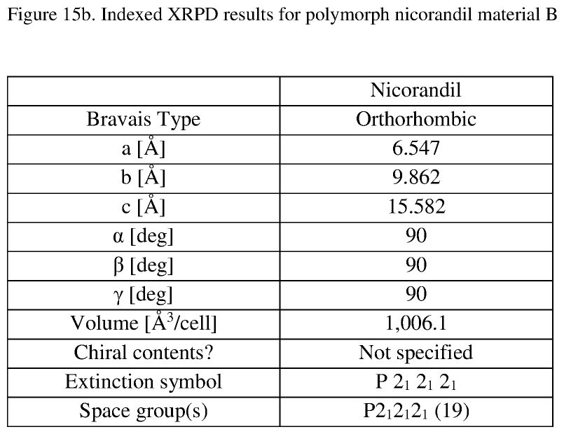

Nicorandil was suspended in zofenopril, which is an oil at ambient temperatures. Heptane was added resulting in the formation of solids. Acetone was added drop-wise leading to reduction of the solids. The solids were triturated at ambient temperature giving rise to a crystalline solid which is a previously unreported form of nicorandil and designated nicorandil material B. The XRPD pattern from nicorandil material B was successfully indexed, indicating it is composed of a single crystalline phase (FIGS. 15a and 15b). It has an orthorhombic unit cell containing four molecules of nicorandil with a formula unit volume of 251.5 Å³ consistent with an anhydrous form.

### Synthesis and Characterization of Nicorandil HCl

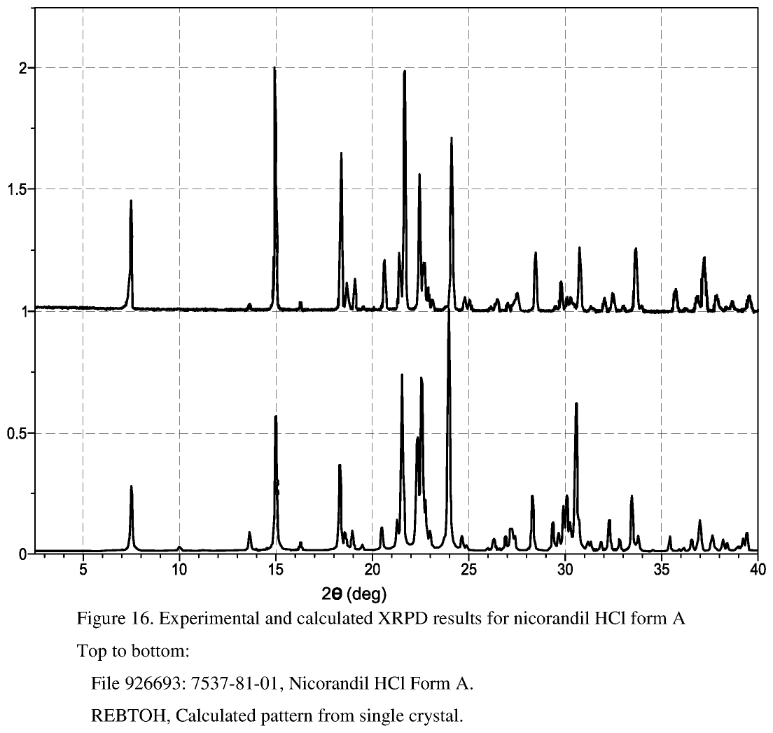

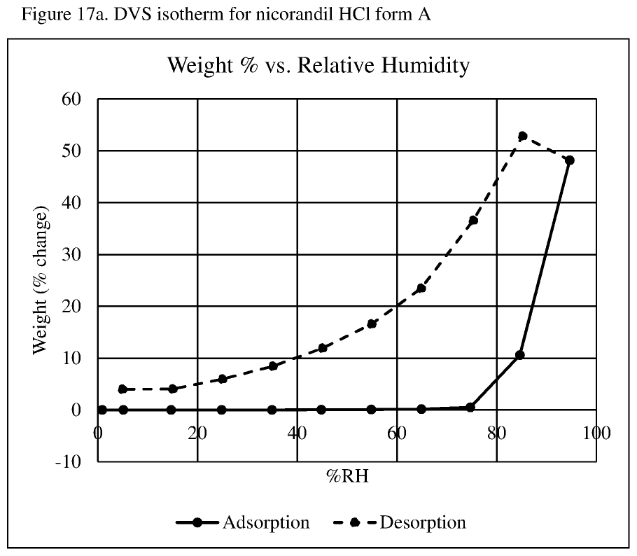

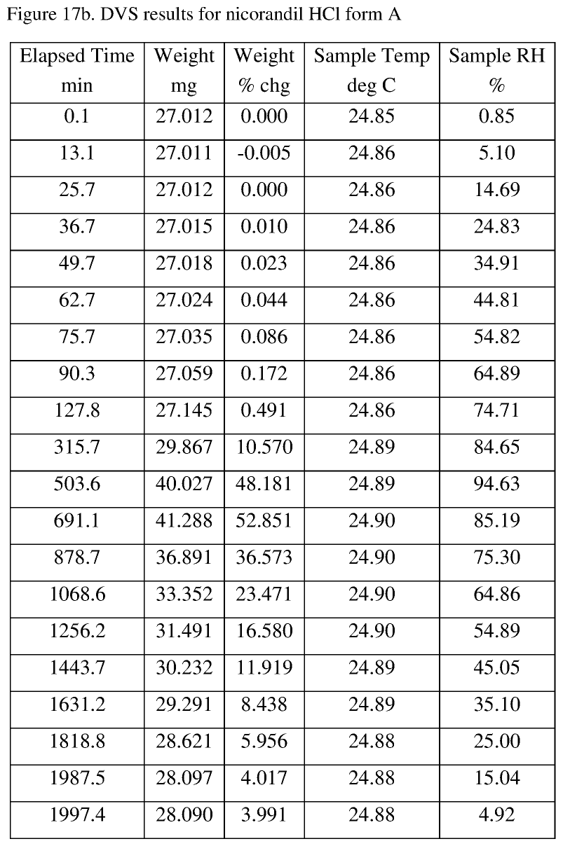

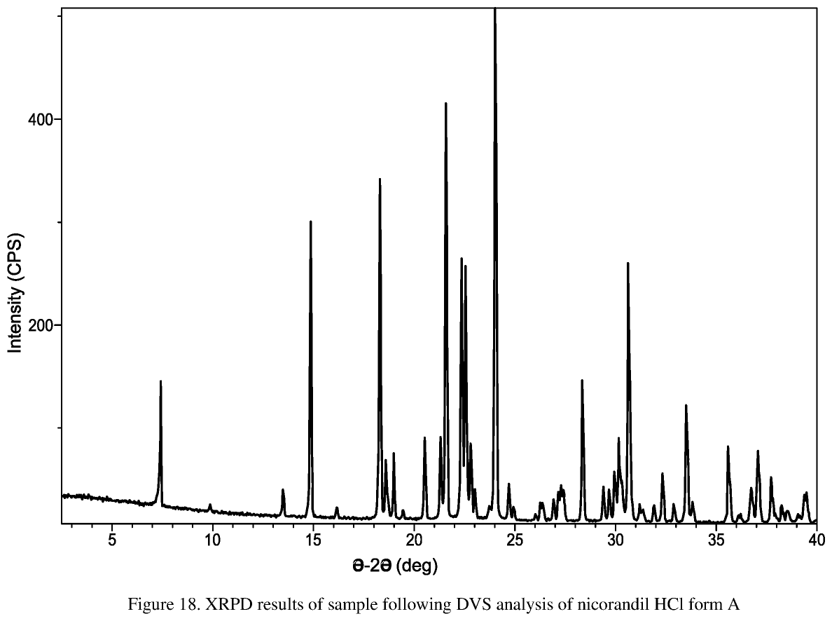

Nicorandil free base was dissolved in acetone resulting in a clear solution. A molar equivalent of HCl was added stepwise following which there nucleation with rosettes of aciculars and blades that were birefringent. The resulting material was recovered and shown to be nicorandil HCl form A. The XRPD pattern for this sample is shown in FIG. 16. Based on the single crystal structure, nicorandil hydrochloride form A is anhydrous and contains one nicorandil cation and one chloride anion in the asymmetric unit. DVS was conducted with the sample and the results are shown in FIGS. 17a and 17b. On the sorption step up to 75% RH, the material gained 0.5% weight. Above that it gained an additional 52% weight. Most of the weight was lost on desoption with significant hysteresis. The material recovered from DVS was shown by XRPD to be form A (FIG. 18). Solubility of nicorandil HCl form A was tested and shown to be >48 mg/mL which was the limit of the test.

In-Vitro Study of Rat Portal Vein Ring Relaxation with Zofenopril and Nicorandil

The portal vein was recovered from Wistar rats weighing approximately 450 gm, and divided into vein segments. Segments were placed in a bath containing approximately 10 mL of Krebs buffer at pH˜7.4. The vehicle used for treatment was 0.2% DMSO. Portal vein rings were pre-contracted and incubated for 60 min at 32° C. Ring relaxation was measured after 10 minutes of exposure to the test compound(s). Chromakalim served as the positive control for the experiment. Ring relaxation was measured by the isometric method as gram changes. Relaxation of the vein rings in this assay are a function of both K_{ATP} channel agonization and NO response. In prior published work, about half of the relaxation response could be attributable to K_{ATP} channel agonization and half to a NO-mediated response. Each assay was run in duplicate.

Based on prior published data using arterial rings, 100 μM zofenopril achieved a maximal relaxation response (J Pharmacol. Exp. Therapeutics; 1993; 265(2):609-618). In a published study with nicorandil using human umbilical artery segments, 10 μM of nicorandil induced relaxation that was about 10% of the maximum, 30 μM induced relaxation that was about 60% of maximum while 100 μM induced maximal relaxation (Eur J Cardio-Thorac. Surg.; 2000; 17:319-324).

Eight combinations of concentration of zofenopril and nicorandil were tested in the assay. These were 80 and 40 μM nicorandil, 100 and 50 μM zofenopril, 12.5 μM zofenopril+10 μM nicorandil, 12.5 μM zofenopril+20 μM nicorandil, 12.5 μM zofenopril+40 μM nicorandil, and 25 μM zofenopril+40 μM nicorandil.

In this assay, 40 μM nicorandil achieved a maximal relaxation response. Consistent with the value reported in the literature, 100 μM zofenopril did achieve a maximal response which was only about 85% of the positive control. Given that 40 μM nicorandil achieved a maximal relaxation response in this assay, the further addition of zofenopril to 40 μM nicorandil could not further increase the relaxation response, which, pooled across the 40 μM nicorandil, 12.5 μM zofenopril+40 μM nicorandil, and 25 μM zofenopril+40 μM nicorandil, was equivalent to 103.6% of the positive control. The remaining combinations show the potential additivity of nicorandil and zofenopril. In this assay, a zofenopril concentration of 50 μM achieved relaxation of 60% of the positive control, or about 70% of the maximum response for zofenopril. The combination of 12.5 μM zofenopril+10 μM nicorandil (equal to concentrations that were ⅛ the maximum for zofenopril and ¼ the maximum for nicorandil) achieved relaxation of 63%, which was equivalent to about 74% of the maximal relaxation response for zofenopril and 61% of the maximal response for nicorandil. Based on additivity of effects, assuming a linear response curve, this combination was expected to have induced relaxation that was approximately 37% of the positive control (equivalent to 43.5% of the maximal response for zofenopril or 36% of the maximal response for nicorandil). Based on the published literature, 10 μM nicorandil alone had a very limited vasorelaxation respone (˜10% of maximum), rather than the value used to calculate the response under the assumption of additivity. Similarly, the combination of 12.5 μM zofenopril+20 μM nicorandil (equal to concentrations that were ⅛ the maximum for zofenopril and ½ the maximum for nicorandil) achieved relaxation that was 92% of the positive control, greater than the maximal response for zofenopril, and 89% of the maximum for nicorandil. Based on additivity of effects, this combination was expected to have induced relaxation that was approximately 62% of the positive control (equivalent to 73% of the maximum for zofenopril or 60% of the maximum for nicorandil). These latter two results show that at substantially submaximal concentrations of nicorandil and zofenopril, the in-vitro response, that comprises both K_{ATP} channel agonization and NO-mediated contributions, is greater than would be predicted by additivity of effects. Put another way, the addition of a low dose of zofenopril, which probably has a very limited direct effect, to low concentrations of nicorandil, substantially and synergistically enhances the therapeutic response over what would be achieved with nicorandil alone. The concentrations tested are in the same range as the circulating drug levels following dosing with nicorandil at 5 mg TID. The Cₘₐₓ following a 5 mg dose is expected to be 15-16 μM, while C_{avg} is substantially lower than that.

1.-117. (canceled)

118. A pharmaceutical coformulation comprising a nitrogen oxide (NO) donor and a hydrogen sulfide (H₂S) releasing agent, wherein the H₂S releasing agent is present in the coformulation in an amount that is effective to enhance the therapeutic efficacy of the NO donor.

119. The pharmaceutical coformulation of claim 118, wherein the NO donor is one or more selected from nicorandil, nitroglycerin, isosorbide mononitrate, pentaerythrityl tetranitrate, sodium nitroprusside, S-nitroso-glutathione, S-nitroso-N-acetylpenicillamine, S-nitroso-N-valerylpenicillamine, an S-nitrosothiol, aspirin, NCX4215, NCX4016, nipradilol, nitropravastatin, SNO-diclofenac, a NO-NSAid, a diazeniumdiolate, SNO-captopril, SNO-t-PA and a S-nitroso hybrid molecule, or a pharmaceutically acceptable salt of any of the foregoing.

120. The pharmaceutical coformulation of claim 118, wherein the H₂S releasing agent is one or more selected from a 1,2-dithiole-3-thione, a 1,2,4-thiadiazolidine-3,5-dione, 4-carboxyphenyl isothiocyanate, ACS-67, ACS-94, an acyl perthiol, AoAA, AP39, an arylthioamide, captopril, CaS, DAS, DADS, DATS, DATS-MSN, a dithioethione glycoconjugate, a dithioperoxyanhydride, DL-propargylglycine, GYY4137, IK-1001, an iminoester, an isothiscyanate glycoconjugate, Lawesson's reagent, a Lawesson's reagent analog, Na₂S, n-acetyl cysteine, NaSH, a N-benzoylthiobenzamide, an O-alkyl phosphorodithioate, an O-aryl phosphorodithioate, S-allylcysteine, a S-aroylthiooxine, SG-1002, S-propylargyl-cysteine, S-propylcysteine, YD0171, and zofenopril, or a pharmaceutically acceptable salt of any of the foregoing.

121. The pharmaceutical coformulation of claim 118, further comprising an angiotensin converting enzyme (ACE) inhibitor.

122. The pharmaceutical coformulation of claim 121, wherein the ACE inhibitor is one or more selected from zofenopril, captopril, enalapril, ramipril, quinapril, perindopril, lisinopril, benazepril, imidapril, trandolapril, cilazapril, and fosinopril, or a pharmaceutically acceptable salt of any of the foregoing.

123. The pharmaceutical coformulation of claim 122, wherein the ACE inhibitor is zofenopril or a pharmaceutically acceptable salt thereof.

124. The pharmaceutical coformulation of claim 118, further comprising a K_{ATP} channel agonist.

125. The pharmaceutical coformulation of claim 124, wherein the K_{ATP} channel agonist is one or more selected from nicorandil, pinacidil, chromakalin, a benzopyran, a cyanoguanidine, a thioformamide, a thiadiazine, a pyridyl nitrate, a cyclobutenedione, a dihydropyridine, a tertiary carbinol, a 3-trifluoromethyl-4-nitro-5-arylpyrazole, a thioamide, a dimethylchroman, a benzothiazole, a tetrahydrobenzothiazole, a benzenesulfonylurea, a benzenesulfonylthiourea, a benzenecarbonylurea, and a benzenecarbonylthiourea, or a pharmaceutically acceptable salt of any of the foregoing.

126. The pharmaceutical coformulation of claim 125, wherein the K_{ATP} channel agonist is nicorandil, or a pharmaceutically acceptable salt thereof.

127. The pharmaceutical coformulation of claim 118, wherein the NO donor is nicorandil, or a pharmaceutically acceptable salt thereof.

128. The pharmaceutical coformulation of claim 118, wherein the H₂S releasing agent is zofenopril, or a pharmaceutically acceptable salt thereof.

129. The pharmaceutical coformulation of claim 118, wherein the nicorandil or a pharmaceutically acceptable salt thereof and zofenopril or a pharmaceutically acceptable salt thereof are present in the coformulation in a molar ratio in the range of about 1:10 to about 5:1.

130. The pharmaceutical coformulation of claim 118, wherein the H₂S releasing agent is present in an amount that is effective to enhance the therapeutic efficacy of the NO donor for reducing the risk of a cardiovascular disease, treating a cardiovascular disease, or both.

131. The pharmaceutical coformulation of claim 118, wherein the H₂S releasing agent is present in the coformulation in an amount that is effective to enhance the therapeutic efficacy of the NO donor for treating a subject having Chronic Kidney Disease (CKD).

132. The pharmaceutical coformulation of claim 131, wherein the subject is also being treated with hemo- or peritoneal dialysis.

133. A method of treating a subject, comprising:
identifying a subject having a chronic kidney disease (CKD); and
administering therapeutically effective amounts of a NO donor and a H₂S releasing agent to the subject.

134. The method of claim 133, further comprising administering an effective amount of an ACE inhibitor to the subject.

135. The method of claim 134, wherein the ACE inhibitor is one or more selected from zofenopril, captopril, enalapril, ramipril, quinapril, perindopril, lisinopril, benazepril, imidapril, trandolapril, cilazapril, and fosinopril, or a pharmaceutically acceptable salt of any of the foregoing.

136. The method of claim 133, further comprising administering an effective amount of a K_{ATP} channel agonist to the subject.

137. The method of claim 136, wherein the K_{ATP} channel agonist is one or more selected from nicorandil, pinacidil, chromakalin, a benzopyran, a cyanoguanidine, a thioformamide, a thiadiazine, a pyridyl nitrate, a cyclobutenedione, a dihydropyridine, a tertiary carbinol, a 3-trifluoromethyl-4-nitro-5-arylpyrazole, a thioamide, a dimethylchroman, a benzothiazole, a tetrahydrobenzothiazole, a benzenesulfonylurea, a benzenesulfonylthiourea, a benzenecarbonylurea, and a benzenecarbonylthioureaor a pharmaceutically acceptable salt of any of the foregoing.
